SEQUENCE LISTING (28 sequences)

SEQ ID NO: 1 - DNA, 1260 bp, synthetic construct
  misc_feature 1..1260: note = Description of Artificial Sequence: Synthetic polynucleotide
  source 1..1260: mol_type = other DNA
ggatccgatctcctttcatttctgataaaagtaaggcttctctatttaccttttaacctacatattcatagttggaagttatccttctaagtacgtatacaatattaattcaacgtaaaaacaaaacttactgtaaatatgtgtaaaaaaaatctattaaattcatggcagtttcaagaaaagaaaactattatggtctggtcacgtgtatacaaattattaattttaaaactatataatttattatttttttattttgaagtttagagtaattttagtagtattttatattttaaataaatatgctttaaatttttacttaatattttattatttttaaatacaacgtttttatttaaaacaaaattataagttaaaaagttgttccgaaagtaaaatatattttatgggttttacaaaaataaattatttttaatgtatttttttaattatatttttgtatgtaattatatccacaggtattatgttgaatttagctgttttagtttacctgtgtggtactatgatttttttagaactctcctcttagaaataggtggtgttgcggttgacttttaacgatatatcattttcaatttatttattttaaagtgacatagagagattccttttaattttttaatttttattttcaataattttaaaaatgggggacttttaaattggaacaaaatgaaaaatatctgttatacgtgcaactgaattttactgaccttaaaggactatctcgaacttggttcggaaatccttgaaatgattgatattttggtggattttctctgattttcaaacaagtagtattttatttaatatttattatattttttacatttttttatatttttttattgtttggaaggtaaagcaacaattactttcaaaatatataaatcaaactgaaatacttaataagagacaaataacattcaagaatcaaatactgggttattaatcaaaagatctctctacatgcgcccaaattcactatttaaatttactataccactgacagaatatatgaacccagattaagtagccagaggctcttccactatattgagtatatagccttacatattttctgcgcataatttactgatgtaaaataaacaaaaatagttagtttgtagttatgaaaaaaggcttttggaaaatgcgaaatacgtgttatttaaggttaatcaacaaaacgcatatccatagtggatagttggataaaacttcaattgatgcggccgc

SEQ ID NO: 2 - DNA, 193 bp, synthetic construct
  misc_feature 1..193: note = Description of Artificial Sequence: Synthetic polynucleotide
  source 1..193: mol_type = other DNA
tcacgtgtatacaaattattaattttaaaactatataatttattatttttttattttgaagtttagagtaattttagtagtattttatattttaaataaatatgctttaaatttttacttaatattttattatttttaaatacaacgtttttatttaaaacaaaattataagttaaaaagttgttccgaaagt

SEQ ID NO: 3 - DNA, 12 bp, synthetic construct
  misc_feature 1..12: note = Description of Artificial Sequence: Synthetic oligonucleotide
  source 1..12: mol_type = other DNA
ttttattgtttg

SEQ ID NO: 4 - DNA, 1205 bp, synthetic construct
  misc_feature 1..1205: note = Description of Artificial Sequence: Synthetic polynucleotide
  source 1..1205: mol_type = other DNA
gatccaagtctgaaggttggtttggcactaactttactcttgttatattcagaattgtatcaagtttatttggtagagtggagcctttttttatccgtaacactttttccctgctccattttgaaaaacgatttcaggccatcttggctattccgaatgaatttggaatatgtttaaattaataaaaataaaataaaataaaataaaataaaataaaaattaaatcaaattaaattaaattaaattaaattaaattaaattaaataaaaataaatacaaccaatacaacatggtaatattcttgcatcgtaatgaatattaaatatcactttattaatctcatcatgttttattgtttttgtaaggactttaatatatttgaatcaatattctttcaattactagtactttttttatatgactaaaattgttacacattggactgacagtaatttttaaaatttatgatttattcttactttatatctttaaaagtagaaatattatacggacgctttgaatacaattgacaacttatcttactagtgtgaatcaaccctatcgatgtagtactcttaaaatacggccttcttgataaagtgttaaattcatttgggtaatgatttttcgaaaaccacattgaatgaacgatctaaataaatataggatgcaaaagcattttaataattcagaaacaaacaaattattaaacaggagcagttgaacggtatgttagcgagttttgtaaagggtgagtacatttatagctctattgaacataataaatacatataaatagtattttttgaccctctatgaagatggcttaccagcaacttatgtcttttaattcacgtgactactaaacaaaaaaatatgttatttaaaaaatatttatttaaatttttaaactattatagattatttgtgaatgcattattttttaatttattaattaaaagaattgctatttacttaaaataagaataaaagctttttatttttttaaaagaaaaatatattaaaaacacttttccgaaagttaaaataattttatatttatcggtagctgcaatttatagacataatattttatattttttaaaatttattattattttgtttgaaataataacgtcggtgagtgtttaaggtgaactaagactgaaaaagtacataatttttgttaattttatgatatgatc

SEQ ID NO: 5 - DNA, 192 bp, synthetic construct
  misc_feature 1..192: note = Description of Artificial Sequence: Synthetic polynucleotide
  source 1..192: mol_type = other DNA
tcacgtgactactaaacaaaaaaatatgttatttaaaaaatatttatttaaatttttaaactattatagattatttgtgaatgcattattttttaatttattaattaaaagaattgctatttacttaaaataagaataaaagctttttatttttttaaaagaaaaatatattaaaaacacttttccgaaagt

SEQ ID NO: 6 - DNA, 12 bp, synthetic construct
  misc_feature 1..12: note = Description of Artificial Sequence: Synthetic oligonucleotide
  source 1..12: mol_type = other DNA
ttttattgtttt

SEQ ID NO: 7 - DNA, 2411 bp, synthetic construct
  misc_feature 1..2411: note = Description of Artificial Sequence: Synthetic polynucleotide
  source 1..2411: mol_type = other DNA
aacagattggtgggtggtcaacgcacaagcgatatcccaacacagtcggaaaaactctcgttcattccaaaactgattgcttcagatcacaactccgctggagaagatgagtccgtcactttctttcaagatttgattaacgttgatcgtttgaaacgtctcagaaatgtcactggtatgtctatcgaaatcgtgcttgggacgcatagagaaatcccacagcaacagcagcagcagcaggagtcacctgtagcagaaggtgttccggtcgcccaggataatggacatgtaaccacgaacgacaatgcggcaaatacttcattggaagaaccaagttcacccattgaccaggtttatggatacctcctacaacagaacatgtctacgttgccagaagttacactttcggaaagtgatatcgctatgagctacccgacggatccagtaccctcttacagcagcaactttaacaactttgctctgcctactattgccgatgacaaacaagaattagaacagatgagattaaaggagctagaaagtgaacctcctatctgaacacttaacgagaaatatttatatgtgtgtttttgtttgtatgtatgtatgtatgtatgcctgtgtatcattaaatatattagcggatcccggagtttttattatcgtgttcttttcattatatagtgaacctaaagtgactttcaattccaaattatggaaagattcctggcattatgccttataataatcacttgtttacaacattccattaacaacacatgtacactcaaattccattccataaaaccaaaaaaaaccttattgaattctccagacctctctgtcggcttgactttgcttgctcaattcgcgtttggctgaagatcactccagaacctaggacgtcattattgaaatctgatcacgtgattcgcatattcatatagacgtatatttttcgccacttttctctcttgaaaaaaagttgtgctagatgaactttgagaacaaaacacattgaaagaaaagtggaacattataataattggaaagaatagtagattgggtggccaagtggaagaatttagtaactttagtggttagagcttgtttgaacgaccaatccagtaaactaatcaaccattgaacaatgagtattcctatctttggagatcaagttaccgaagagagagcagaaaatgctcgtatgagtgcctttgttggtgccatcgccgttggtgatctagtgaaaactacactaggtccaaaaggtatggataagttacttcaaagtgcatccaatagctcgagtttggttacaaacgatggtgctaccattctaaaatctattcctttggacaaccctgctgccaaggtgcttgttaacatcagtaaagtgcaagatgatgaagttggtgacggtacaacaagtgttactgttctaagtgcagaattattgagggaagctgaaaaacttgttgaacaaggcagaattcacccacaaactatcatcgagggttacagaattgcttctgctgctgccctctctgcattggaaaaggctgctgtggacaactccaagaataaagaagaattttacaatgatttgatcagcatcgccaacacaacgctatcttctaaaattctatctcaagataaggctcacttctctaagttggctaccgatgctatcttaagattaaagggctctacgaacttggaacacattcaaattattaagatcattggtggtaaattatcggattctttcctagatgaaggtttcattttgccaaagagatttggtaccaaccaaccaaaacgtgttgaaaatgcgaagattttgattgccaacacttctctagatacagacaaggttaaaatctttggtaccaaatttaaggtcgactctacttccaagttagctgaactagaaaaagctgagcgtgaaaaaatgaagagaaagatagaaaagattgcacaattcaacattaatacctttatcaacagacaattaatctatgactaccctgaacagatgtttaccgacatgggtatcaactccatcgaacatgctgactttgaaggtgttgaaagattagcacttgtcactggcggtgaggttgtttctacatttgacaacccagaaaaatgtaagctaggtgaatgtaagttgatcgaagaagttataattggtgaggaaatctttactaaatttaccgggtgcaagtctggtgaagcttgtaccattgttctaaggggtgccactgagcaagtcttggatgaagcagaaagatctctacatgatgccctatctgttctttcccaaacaacaaaggagactagaaccgttcttggtggtggttgtgcagaaatgataatgtctaaagcagttgatactgcagctcaaa

SEQ ID NO: 8 - DNA, 529 bp, synthetic construct
  misc_feature 1..529: note = Description of Artificial Sequence: Synthetic polynucleotide
  source 1..529: mol_type = other DNA
actttcggaaagtgatatcgctatgagctacccgacggatccagtaccctcttacagcagcaactttaacaactttgctctgcctactattgccgatgacaaacaagaattagaacagatgagattaaaggagctagaaagtgaacctcctatctgaacacttaacgagaaatatttatatgtgtgtttttgtttgtatgtatgtatgtatgtatgcctgtgtatcattaaatatattagcggatcccggagtttttattatcgtgttcttttcattatatagtgaacctaaagtgactttcaattccaaattatggaaagattcctggcattatgccttataataatcacttgtttacaacattccattaacaacacatgtacactcaaattccattccataaaaccaaaaaaaaccttattgaattctccagacctctctgtcggcttgactttgcttgctcaattcgcgtttggctgaagatcactccagaacctaggacgtcattattgaaatctgatcacgtga

SEQ ID NO: 9 - DNA, 10 bp, synthetic construct
  misc_feature 1..10: note = Description of Artificial Sequence: Synthetic oligonucleotide
  source 1..10: mol_type = other DNA
aaacaacaaa

SEQ ID NO: 10 - DNA, 1583 bp, synthetic construct
  misc_feature 1..1583: note = Description of Artificial Sequence: Synthetic polynucleotide
  source 1..1583: mol_type = other DNA
tcaattacaaagggtggaaagtgatggggggaatatcatctgcacaattttggctcgctttatatagtgccgagattagtagggtctggataaaaaagcgaaggagaataggaagaggaagaaaattttttttcttcctctttgaaaggccgggtaacaaagtctcatcgtcctccaacctagggctttcctttccgctttttttttcttcttctcctccaaacaagacccaaccatacacacccacacagacagaagaaaaagtgtaaggatgagcgttgtgtcgttttttttttttttttttttttttttggcggagaatgtgtgcacgtgcacagacacacacgggagcggctgtgcctccgtatacggcaactgccacgacaaccgagggcacagatacacgaggttatgtcaaagaggcgtgctggcctgggggggggaggctgcggatgcctgatactggggcctgatactgagccccaaggctcagtctcggtctctgtctcaagctcaagccaattccttccggggaacccaaccacctccggattttttccgaaagtatccccgaacgtctatggattatccatgtatacacagaacagggagtgagtgagtgagtgcgaaaaacgaaaaaaaatacagtaaaacataaaccagagatagcagggaaaagagccgtggtgcggcgcactgcgcgccgccctggggacggcgcctctctctagttcccccagaaaaaagagtcacgtgtacacagccgcagccgcagccgcagccgcagtatctccgtgtcacatagattggactgaactggactagactagactagactagagagtagacgagaatagacgagactagacgctctggcgtttcagataacaccaacactatctatgttatcattacacacacgatacgtaatacgttggggctccagcggtcaaggttgggggtgtggcccacatacgtaacgtctcgccctacaccatacacggcatttttgtctgcctgccggctttggcttgcgctttggtacttggtattttttcctctttctttttgtttccaccttcaacagacatctacgcttttacagttcaagacattgaaatttcaagactagaactagaattagaaattggaaatgaaattggaattataatagatattagaaatagatagatattagaatagagatagatattcgagtaatagaaaggacaaaagtcaggaagaagaaaacttagggcgagcgaagctgccgtattaatctattggaaaactgaaatactaggtttcagagaagaagaacaaacaaaaagcgcaataaccagcactttatccaagttacaagtgtgagtgagtgtatatctgcaagcaaggtgtgattgagtgagtgatccgcttgtgatggattctgtcgctgatagcacccttgtttccaaagctgtagcacagccttcgccgcatcatgctgtgataaagcgtgaacatgagcaggaaagagaaagacaaatagaagccgaagcagaggcagaagcagaggcagaagcagaagcagaaacagaaataga

SEQ ID NO: 11 - DNA, 200 bp, synthetic construct
  misc_feature 1..200: note = Description of Artificial Sequence: Synthetic polynucleotide
  source 1..200: mol_type = other DNA
ttccgaaagtatccccgaacgtctatggattatccatgtatacacagaacagggagtgagtgagtgagtgcgaaaaacgaaaaaaaatacagtaaaacataaaccagagatagcagggaaaagagccgtggtgcggcgcactgcgcgccgccctggggacggcgcctctctctagttcccccagaaaaaagagtcacgtg

SEQ ID NO: 12 - skipped

SEQ ID NO: 13 - DNA, 1256 bp, synthetic construct
  misc_feature 1..1256: note = Description of Artificial Sequence: Synthetic polynucleotide
  source 1..1256: mol_type = other DNA
gagctcctttcatttctgataaaagtaaggtttctctatttatcttttcacccacattatccttcgaagtacgtatacaatattagttcaacgtaaaaacaaaacttactgtaaatatgcgtaaaaaaaatctattaaattcatagcagtttcaaggaaagagaaccattatggtctggtcacgtgtgtataaattattaattttaacactatataatttattatttttttattttgaagtttagagtaattttagtagtattttatattttaaataaatatattttaaatttttacttaatattttattattttttaatacaatgtttttatttaaaacaaaattataaattaaaatgttgttccgaaagtaaaatatattttatggtttttacaaaaataaattatttttaatgtatttttttaattatatttttgtatgtaattatatccacaggtattatgttgaatttagctgttttagtttacctgtgtggtactatgagttttttgcctctcaaaagctattttttagaactctctctcctcttagaaataggtggtgttgcggttgacttttaacgatatatcattttcaatttatttattttaaagtgacatagagagattccttttaattttttaatttttattttcaataattttaaaaatgggggacttttagattggaacaaaatgaaaaatatctgttatacgtgcaactgaattttactgaccttaaaggactatctcgaacttggttcggaaatccttgaaatgattgatattttggtggattttctctgattttcaaacaagtagtattttatttaatatttattatattttttacatttttttatatttttttattgtttggaagggaaagcaacaattactttcaaaatatataaattaaactgaaatacttaataagagacaaataacattcaagaatcaaatactgggttattaatcaaaagatctctctacatgcacccaaattcactatttaaatttactataccactgacagaatatatgaacccagattaagtagccagaggctcttccactatattgagtatatagccttacatattttctgcgcataattttctggatgtaaaataaacaaaaatagttagtttgtagttatgaaaaaaggcttttggaaaatgcggaatacgtgttatttaaggttaatcaacaaaacgcatatccatagtggatagttggataaaacttcaattgat

SEQ ID NO: 14 - DNA, 232 bp, synthetic construct
  misc_feature 1..232: note = Description of Artificial Sequence: Synthetic polynucleotide
  source 1..232: mol_type = other DNA
gtcccaggtctctacagtgaaaatatttgctaattgcatacaggaggcttaactatctccgttatataaaaatatgaacacccttttaaaacagttgctgtcaactaaatttagaatgttttttcactttggatgaactttttaatgtgatccactagttttaattaaatatgattggaaagcacttttccgtaacaaaatgatacaaaatggtcaatgttagaaagtactg

SEQ ID NO: 15 - DNA, 19 bp, synthetic construct
  misc_feature 1..19: note = Description of Artificial Sequence: Synthetic oligonucleotide
  source 1..19: mol_type = other DNA
cacgggcagcgcggggtcg

SEQ ID NO: 16 - DNA, 19 bp, synthetic construct
  misc_feature 1..19: note = Description of Artificial Sequence: Synthetic oligonucleotide
  source 1..19: mol_type = other DNA
gaatcccccagaccacact

SEQ ID NO: 17 - DNA, 19 bp, synthetic construct
  misc_feature 1..19: note = Description of Artificial Sequence: Synthetic oligonucleotide
  source 1..19: mol_type = other DNA
gcagttcccttggcgtact

SEQ ID NO: 18 - DNA, 19 bp, synthetic construct
  misc_feature 1..19: note = Description of Artificial Sequence: Synthetic oligonucleotide
  source 1..19: mol_type = other DNA
ggccgcgggcaacagcccg

SEQ ID NO: 19 - DNA, 19 bp, synthetic construct
  misc_feature 1..19: note = Description of Artificial Sequence: Synthetic oligonucleotide
  source 1..19: mol_type = other DNA
gtccgcccagcacagcgca

SEQ ID NO: 20 - DNA, 19 bp, synthetic construct
  misc_feature 1..19: note = Description of Artificial Sequence: Synthetic oligonucleotide
  source 1..19: mol_type = other DNA
gcccggctcaaaaccgccc

SEQ ID NO: 21 - DNA, 7254 bp, synthetic construct
  misc_feature 1..7254: note = Description of Artificial Sequence: Synthetic polynucleotide
  source 1..7254: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgccggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagcctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgcagttcccttggcgtactcgttttagagctagaaatagcaagttaaaataaggctagtccgttatcaacttgaaaaagtggcaccgagtcggtggtgctttttttgttttttatgtctcagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagggatccgatctcctttcatttctgataaaagtaaggcttctctatttaccttttaacctacatattcatagttggaagttatccttctaagtacgtatacaatattaattcaacgtaaaaacaaaacttactgtaaatatgtgtaaaaaaaatctattaaattcatggcagtttcaagaaaagaaaactattatggtctggtcacgtgtatacaaattattaattttaaaactatataatttattatttttttattttgaagtttagagtaattttagtagtattttatattttaaataaatatgctttaaatttttacttaatattttattatttttaaatacaacgtttttatttaaaacaaaattataagttaaaaagttgttccgaaagtaaaatatattttatgggttttacaaaaataaattatttttaatgtatttttttaattatatttttgtatgtaattatatccacaggtattatgttgaatttagctgttttagtttacctgtgtggtactatgatttttttagaactctcctcttagaaataggtggtgttgcggttgacttttaacgatatatcattttcaatttatttattttaaagtgacatagagagattccttttaattttttaatttttattttcaataattttaaaaatgggggacttttaaattggaacaaaatgaaaaatatctgttatacgtgcaactgaattttactgaccttaaaggactatctcgaacttggttcggaaatccttgaaatgattgatattttggtggattttctctgattttcaaacaagtagtattttatttaatatttattatattttttacatttttttatatttttttattgtttggaaggtaaagcaacaattactttcaaaatatataaatcaaactgaaatacttaataagagacaaataacattcaagaatcaaatactgggttattaatcaaaagatctctctacatgcgcccaaattcactatttaaatttactataccactgacagaatatatgaacccagattaagtagccagaggctcttccactatattgagtatatagccttacatattttctgcgcataatttactgatgtaaaataaacaaaaatagttagtttgtagttatgaaaaaaggcttttggaaaatgcgaaatacgtgttatttaaggttaatcaacaaaacgcatatccatagtggatagttggataaaacttcaattgatgcggccgcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatattaagaaaccattattatcatgacattaacctataaaaataggcgtatcacgaggccctttcgtc

SEQ ID NO: 22 - DNA, 7500 bp, synthetic construct
  misc_feature 1..7500: note = Description of Artificial Sequence: Synthetic polynucleotide
  source 1..7500: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcggaataaaaaacacgctttttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgactaagttgtattctgacttgtacaggacctgcatgacatgcggagaagaaaaattgtcaaccgagttctacgtcaggaacaagaagaccggagttagacattcatcatgcaaagagtgtgacaaggtcagggtcaaatcaagacacaaggagaaccctgaaaggaccaaaaacaacgacttgaagagattgtacggaatcaccttggacgagcatacccaaatgtatgaggaacaaaatggtgtatgtgcaatttgcaagggagaaggagatggaaagtggaagaaattgtgtgttgaccatgatcacgaaacaggaaaggtcaggcagttgttgtgtaggaactgcaatatgatgttgggtcaggtcaacgacaacgttaacttattatcagaaatgataaagtatttgaaaagatatcagtaaaacctgcaggccgcgagcgccgattaagtgaatttactttaaatcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttaacgcgatcgccgacgccgccgatgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggctcgaggatctcctttcatttctgataaaagtaaggcttctctatttaccttttaacctacatattcatagttggaagttatccttctaagtacgtatacaatattaattcaacgtaaaaacaaaacttactgtaaatatgtgtaaaaaaaatctattaaattcatggcagtttcaagaaaagaaaactattatggtctggtcacgtgtatacaaattattaattttaaaactatataatttattatttttttattttgaagtttagagtaattttagtagtattttatattttaaataaatatgctttaaatttttacttaatattttattatttttaaatacaacgtttttatttaaaacaaaattataagttaaaaagttgttccgaaagtaaaatatattttatgggttttacaaaaataaattatttttaatgtatttttttaattatatttttgtatgtaattatatccacaggtattatgttgaatttagctgttttagtttacctgtgtggtactatgatttttttagaactctcctcttagaaataggtggtgttgcggttgacttttaacgatatatcattttcaatttatttattttaaagtgacatagagagattccttttaattttttaatttttattttcaataattttaaaaatgggggacttttaaattggaacaaaatgaaaaatatctgttatacgtgcaactgaattttactgaccttaaaggactatctcgaacttggttcggaaatccttgaaatgattgatattttggtggattttctctgattttcaaacaagtagtattttatttaatatttattatattttttacatttttttatatttttttattgtttggaaggtaaagcaacaattactttcaaaatatataaatcaaactgaaatacttaataagagacaaataacattcaagaatcaaatactgggttattaatcaaaagatctctctacatgcgcccaaattcactatttaaatttactataccactgacagaatatatgaacccagattaagtagccagaggctcttccactatattgagtatatagccttacatattttctgcgcataatttactgatgtaaaataaacaaaaatagttagtttgtagttatgaaaaaaggcttttggaaaatgcgaaatacgtgttatttaaggttaatcaacaaaacgcatatccatagtggatagttggataaaacttcaattgatgaattc

SEQ ID NO: 23 - DNA, 8090 bp, synthetic construct
  misc_feature 1..8090: note = Description of Artificial Sequence: Synthetic polynucleotide
  source 1..8090: mol_type = other DNA
gacggcacggccacgcgtttaaaccgccgttatagatgatctttatgactatcacataaattttgtaacctttttcattggttcaaaggttaaaccatttgatgacacgacattcgcagatatcttaaggtggggatcaaaagttaatgacactaaaaattggttatattctcatggtccaaatacaaaacccataaatgcatcgactattaagtctaaggtcaaaaggacaaaggaaataaatagagtgaaatttcgctgtcctttaatattagacgaaagagcagactttgttgtttctgttagtggatacacaattatatctcatgagattcctgcatcgaaatacaagcttatatatgataatggtacggtcaaacaagaagcgtactcccgtcgtgaatatcttgatgcggaaactggagaagtggtaccaaatgacgaacttgcaaaaaccttttcatttggagatgaaataattgagttgtctgaggaagagaactcacaaattcaaaacatatacggaaattatgactcatttttgaagctaataggatttagatctaccgaggaatgcttatgtttttacaataatatcgacgctcgtccaacgccggcggacctcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaaacctcccgcgacctccaaaatcgaactaccttcacaatggataagaaatactctatcggtttggatattggtactaactccgttggttgggccgttatcactgatgaatacaaggttccatctaagaagttcaaagttttgggtaacactgatagacactctatcaagaagaacttgattggtgctttgttatttgactctggtgaaaccgctgaggctacccgtttaaaaagaactgctagacgtagatacacccgtcgtaaaaacagaatctgttatttgcaagagatcttctccaacgaaatggctaaggttgacgactcttttttccatagattagaagaatctttcttagttgaagaagataagaagcacgaacgtcatccaatcttcggtaacattgtcgacgaagttgcttaccatgaaaagtacccaactatctatcacttgagaaagaaattggttgattctactgacaaagccgacttgagattgatctacttggctttagctcatatgatcaaattccgtggtcattttttaattgaaggtgatttgaacccagacaactctgacgttgataaattgttcatccaattggttcaaacctataaccaattgtttgaagaaaacccaattaacgcttctggtgttgatgctaaggctatcttgtctgctagattgtctaaatctagaagattggaaaacttaattgctcaattgccaggtgaaaaaaaaaacggtttgttcggtaatttgattgctttatccttgggtttgaccccaaatttcaagtccaactttgatttggctgaagatgccaagttgcaattgtctaaggatacttacgatgatgatttagataacttattggctcaaattggtgatcaatacgctgatttgtttttagctgccaagaatttgtccgacgccattttgttgtctgacatcttgagagtcaacactgaaattaccaaggcccctttgtctgcttctatgattaagagatatgacgaacaccaccaagacttgaccttgttgaaggctttggttagacaacaattacctgaaaagtataaggaaatttttttcgaccaatctaagaacggttacgctggttacattgacggtggtgcctctcaagaagaattctacaaattcatcaaaccaatcttggaaaagatggacggtactgaagaattgttagttaaattgaacagagaagacttgttgagaaaacaaagaacctttgacaacggttccattcctcaccaaatccacttgggtgagttacacgctattttgagaagacaagaagatttctacccattcttaaaggacaaccgtgaaaagattgaaaagattttgaccttcagaattccatactacgtcggtcctttggctcgtggtaactccagattcgcctggatgactagaaagtccgaagaaactattactccatggaacttcgaagaagtcgttgacaagggtgcttctgctcaatcctttatcgaaagaatgaccaacttcgacaaaaacttgccaaacgaaaaagtcttgccaaagcactctttgttgtatgaatactttactgtttataatgaattgactaaagttaagtacgttactgaaggtatgagaaaaccagcttttttatctggtgaacaaaaaaaagctatcgtcgatttgttgttcaaaactaaccgtaaagttaccgtcaagcaattgaaggaagattacttcaagaagattgaatgttttgactccgtcgaaatctccggtgttgaagacagattcaatgcttctttgggtacttaccacgacttgttgaaaattatcaaggacaaggatttcttagataacgaagaaaacgaagacattttggaagatattgtcttgactttgactttgttcgaagatagagaaatgattgaagaaagattgaagacttatgctcatttgttcgacgataaggtcatgaagcaattaaagagaagacgttacactggttggggtagattgtctagaaaattgattaacggtatccgtgataaacaatctggtaagaccatcttggatttcttaaagtctgatggttttgccaacagaaacttcatgcaattgatccacgacgactctttgactttcaaggaggacattcaaaaggctcaagtttctggtcaaggtgactctttgcatgaacacattgccaacttggctggttctccagctattaagaagggtatcttgcaaactgttaaggttgttgatgaattagttaaggtcatgggtagacacaagccagaaaacatcgtcatcgaaatggctagagaaaaccaaactactcaaaagggtcaaaagaattctagagaaagaatgaagagaattgaggaaggtattaaggaattaggttcccaaattttgaaggaacatccagtcgaaaacactcaattgcaaaacgaaaaattgtacttgtactacttacaaaacggtagagatatgtatgtcgaccaagagttggacatcaacagattgtccgactacgatgttgatcacatcgttccacaatccttcttaaaggacgactctatcgacaacaaggtcttaaccagatccgacaaaaacagaggtaagtctgacaacgttccatccgaagaagttgttaaaaagatgaagaactactggagacaattgttgaacgccaaattgatcactcaaagaaagttcgataatttgaccaaggctgaaagaggtggtttgtctgaattggataaggctggttttattaaaagacaattggttgagactagacaaatcaccaagcatgtcgctcaaattttagattccagaatgaacactaaatacgacgaaaacgataagttaattagagaagttaaggttattaccttgaagtctaagttggtttctgatttcagaaaggacttccaattttacaaggtcagagaaattaacaactaccatcacgctcatgatgcttacttgaacgccgttgttggtaccgctttgattaaaaagtacccaaagttggaatccgaatttgtctacggtgactacaaggtctacgatgtcagaaaaatgatcgctaagtccgaacaagagattggtaaggctactgccaagtacttcttttactctaacatcatgaactttttcaagactgaaatcactttagctaacggtgaaattcgtaagagaccattgattgaaaccaacggtgagactggtgaaatcgtttgggataagggtcgtgatttcgctactgttagaaaggtcttatctatgccacaagttaacatcgtcaagaaaaccgaagttcaaactggtggtttttctaaggaatctatcttgccaaaaagaaactctgataaattgattgctagaaagaaggattgggacccaaagaagtacggtggtttcgattccccaaccgtcgcttactccgtcttggttgtcgctaaagttgaaaagggtaagtccaagaaattgaagtctgttaaggaattgttgggtatcactatcatggaaagatcttccttcgaaaagaacccaatcgattttttagaggccaagggttataaggaagttaaaaaggacttaattattaagttgccaaagtactctttgttcgaattagaaaacggtagaaaaagaatgttggcctctgctggtgagttgcaaaaaggtaacgaattggccttgccatctaagtatgttaactttttgtacttggcctctcattacgagaagttgaagggttccccagaagataacgaacaaaagcaattgttcgtcgaacaacacaaacattacttggatgaaattatcgaacaaatctccgagttttccaaacgtgttatcttggctgacgccaatttggataaggttttgtctgcttataataagcatagagataagccaattagagaacaagccgagaacatcattcacttgttcactttgactaatttaggtgctccagctgccttcaaatatttcgacaccaccattgatagaaagagatacacctccactaaggaagtcttggatgccaccttgattcaccaatctatcactggtttgtacgaaactagaatcgatttgtctcaattaggtggtgattcccgtgccgacccaaagaagaagagaaaggtctaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttggcatccccgcgtgcttggccggccgtttaatcagcgcccagagactagcactgaatgatcaacgggtagttcacacgatgcacgagcgcaacgctcacaatgacagtctggacatcaatagtcacactacagaaggtgatctctcaacttcagcagaccatagcgtgtaataaatgcataattatttttctctaaaaaaaactcagctgaaattttatataagtactacattttatatacatattacatactgaacaataagcgcgtttgacattttaattttcgaagaccgcgaatccttacatcacacccagtcccccaatagttcccccacacaccatgcttcaaaaacgcactgtactcctttttactcttccggattttctcggactctccgcatcgccgcacgagccaagccacacccacacacctcataccatgtttcccctctttgactctttcgtgcggctccattacccgcatgaaactgtataaaagtaacaaaagactatttcgtttctttttctttgtcggaaaaggcaaaaaaaaaaatttttatcacatttctttttcttgaaaattttttttgggattttttctctttcgatgacctcccattgatatttaagttaataaaaggtctcccgttttccaagttttaatttgttcctcttgtttagtcattcttcttctcagcattggtcaattagaaagagagcatagcaaactgatctaagttttaattaccatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggaaattgataagacttttctagttgcatatcttttatatttaaatcttatctattagttaattttttgtaatttatccttatatagtctggttattctaaaatatcatttcagtatctaaaatagttctttttttttttgagttagatttttatgggggagagttgaagtgttgaattttcccactttgcttcgggattgtgggtcattctgtcgataactgatatcacatcatcaatagaacctcttagatgcacgagcgcaacgctcacaattaatcagcgcccagagactagcactgaatgatcaacgggtagttcacacaggtccgccggcgttggacgagcgctatcgtataccatttatagatgaagtcaggaaactacctactttatcgagctatccagaactactagaaagtgatgattatcaagtactcagtagagtcactgaaacgctcgtgaattttttcaatttgaaaaatgggtacaagccttctgattaccacagcccagcgcttcaaagacacttcacggtactcagagagtatcttctccagattgaaagtaaggaaactaaagatcaagatgaagatgacgaaactcttctgaaagtcaaacagattcacgaaagaattgctgcttctgctcaatcagatgatcctaaacagcaaagactagtaaagtatttgaaactatggaattcatattacaatcgctataataatttggaaattgaatcaaaaccaaaacagaataaacggagtaaatttaatatataatatataataatattctatcggcggtttaaacgcgtggccgtgccgtc

SEQ ID NO: 24 - DNA, 32 bp, synthetic construct
  misc_feature 1..32: note = Description of Artificial Sequence: Synthetic oligonucleotide
  source 1..32: mol_type = other DNA
ccttataaatcaaaagaatagaccgagatagg

SEQ ID NO: 25 - DNA, 22 bp, synthetic construct
  misc_feature 1..22: note = Description of Artificial Sequence: Synthetic oligonucleotide
  source 1..22: mol_type = other DNA
tgttgtgtggaattgtgagcgg

SEQ ID NO: 26 - DNA, 35 bp, synthetic construct
  misc_feature 1..35: note = Description of Artificial Sequence: Synthetic oligonucleotide
  source 1..35: mol_type = other DNA
gttttagagctagaaatagcaagttaaaataaggc

SEQ ID NO: 27 - DNA, 25 bp, synthetic construct
  misc_feature 1..25: note = Description of Artificial Sequence: Synthetic oligonucleotide
  source 1..25: mol_type = other DNA
cgatcatttatctttcactgcggag

SEQ ID NO: 28 - protein, 9 aa, unidentified
  REGION 1..9: note = Description of Unknown: 'LAGLIDADG' family motif peptide
LAGLIDADG

(19) United States
(12) Patent Application Publication
(10) Pub. No.: US 2024/0182927 A1
(43) Pub. Date: Jun. 6, 2024

(54) METHODS FOR GENOMIC INTEGRATION FOR KLUYVEROMYCES HOST CELLS

(71) Applicant: Amyris, Inc., Emeryville, CA (US)

(72) Inventor: Jessica WALTER, Albany, CA (US)

(21) Appl. No.: 18/199,278

(22) Filed: May 18, 2023

Related U.S. Application Data
(62) Division of application No. 16/646,028, filed on Mar. 10, 2020, now Pat. No. 11,685,934.
(60) Provisional application No. 62/560,029, filed on Sep. 18, 2017.
(60) Provisional application No. 62/667,000, filed on May 4, 2018.

(51) Int. Cl. C12N 15/90; C12N 9/22; C12N 15/11; C12N 15/81

(52) U.S. Cl. CPC C12N 15/905; C12N 9/22; C12N 15/11; C12N 15/815; C12N 2310/20; C12N 2800/80

(57) ABSTRACT

The present invention provides high efficiency targeted and marker-less single or simultaneous multiple integrations using nucleases and a stable plasmid in Kluyveromyces host cells.

### 1. CROSS-REFERENCES TO RELATED APPLICATIONS

The present application is a divisional of U.S. application Ser. No. 16/646,028 filed Mar. 10, 2020, which is a U.S. National Phase Application Under 371 of PCT/US2018/050732 filed Sep. 12, 2018, which claims priority to U.S. Provisional Application Nos. 62/560,029, filed Sep. 18, 2017, and 62/667,000, filed May 4, 2018, the disclosures of which are incorporated herein in their entireties.

### STATEMENT AS TO RIGHTS TO INVENTIONS MADE UNDER FEDERALLY SPONSORED RESEARCH AND DEVELOPMENT

This invention was made with Government support under Agreement HR0011-15-3-0001, awarded by DARPA. The Government has certain rights in the invention.

### 2. REFERENCE TO SUBMISSION OF A SEQUENCE LISTING AS A TEXT FILE

The Sequence Listing written in file 101928-1358662 000530SL.xml created on Sep. 18, 2023, 68,158 bytes, machine format IBM-PC, MS-Windows operating system, is hereby incorporated by reference in its entirety for all purposes.

### 3. FIELD OF THE INVENTION

The methods and compositions provided herein generally relate to the fields of molecular biology and genetic engineering.

### 4. BACKGROUND

Genetic engineering techniques to introduce targeted modification into a host cell genome find use in a variety of fields. Fundamentally, the determination of how genotype influences phenotype relies on the ability to introduce targeted insertions or deletions to impair or abolish native gene function. In the field of synthetic biology, the fabrication of genetically modified microbes capable of producing compounds or proteins or interest requires the insertion of customized DNA sequences into a chromosome of the host cell; industrial scale production generally requires the introduction of multiple genes in a host cell genome.

For certain host cells, particularly for conventional yeast cells (e.g., Saccharomyces cerevisiae), genetic tools are well developed to perform targeted genomic gene deletions and integrations. A variety of non-conventional yeast cells are attractive hosts for industrial applications (e.g., small molecule and protein production). However, the tools for engineering these species are generally poor. For example, the genus Kluyveromyces, in particular K. marxianus, is an attractive yeast host for the production of industrial products and antibodies due to its fast growth, high acid tolerance and high temperature tolerance. However, making targeted genomic changes to K. marxianus has been historically time-consuming due to a high basal rate of non-homologous end joining (NHEJ) and difficulty of maintaining a stable plasmid. CRISPR-based disruption of genes in K. marxianus was recently reported for the first time, but no genes were integrated and disruption relied upon NHEJ. Currently, neither meganuclease-mediated, targeted, single genomic integrations nor multiplexed integrations have been reported in K. marxianus. Such integrations would dramatically reduce the genetic engineering cycle time by at least 50%.

Therefore, currently known methods for genomic modification for various Kluyveromyces host cells are in need for improvement. The present invention addresses these and other needs.

### 5. SUMMARY

The present invention provides methods of modifying a target site in a Kluyveromyces host cell genome. The methods comprise contacting the host cell, which has reduced non-homologous end joining (NHEJ) activity, with: a first linear nucleic acid capable of homologous recombination with itself or with one or more additional linear nucleic acids contacted with the host cell, whereby homologous recombination in the host cell results in formation of a circular extrachromosomal nucleic acid comprising a coding sequence for a selectable marker and a stability element from K. marxianus. In some embodiments, the stability element comprises a CEN sequence at least 95% identical to SEQ ID NO: 2 and an ARS consensus sequence at least 90% identical to SEQ ID NO: 3; a nuclease capable of cleaving the target site; and a donor DNA molecule capable of homologous recombination at the cleaved target site, whereby homologous recombination in the host cell results in integration of the donor linear nucleic acid at the target site. Transformed host cells expressing the selectable marker are then selected. In some embodiments, the methods further comprise recovering a host cell wherein the donor DNA molecule has homologously recombined at the target site.

In some embodiments the stability element is at least 90% identical to a sequence less than 750 bp in length and comprising residues 202 to 876 or residues 537 to 1252 of SEQ ID NO: 1 In other embodiments, the stability element is at least 95% identical to SEQ ID NO: 1. The host cell can be K. marxianus.

In some embodiments, the step of contacting includes contacting the host cell with two or more donor DNA molecules capable of homologous recombination with different target sites in the host cell genome, whereby homologous recombination in the host cell results integration of the donor DNA molecules at the different target sites.

In some embodiments, the circular extrachromosomal nucleic acid further comprises a coding sequence for the nuclease. The nuclease in some cases may be an RNA-guided DNA endonuclease, as a Cas9 endonuclease.

In some embodiments, the circular extrachromosomal nucleic acid further comprises a sequence that encodes a crRNA activity and a tracrRNA activity that enables site-specific recognition and cleavage of the target site by the RNA-guided DNA endonuclease. The crRNA activity and the tracrRNA activity may be expressed as a single contiguous RNA molecule.

In some embodiments, the nucleic acid encoding the RNA-guided DNA

endonuclease is pre-integrated into the host cell genome prior to contacting the host cell with sequences that encode a crRNA activity and a tracrRNA activity. The NHEJ may be reduced by integrating the nucleic acid encoding the RNA-guided endonuclease at YKU70 or YKU80 loci. The invention also provides method for modifying a target site in a Kluyveromyces host cell genome, these methods comprise: contacting the host cell, which has reduced non-homologous end joining (NHEJ) activity, with a nucleic acid molecule comprising a stability element comprising a CEN sequence at least 95% identical to SEQ ID NO: 2 and an ARS consensus sequence at least 90% identical to SEQ ID NO: 3 and nucleic acid sequence encoding a nuclease capable of cleaving the target site; and a donor DNA molecule capable of homologous recombination at the cleaved target site. A transformed host cell is then selected in which the donor DNA molecule integrated into the target site. The host cell may be K. marxianus. The nuclease may be a meganuclease, as F-CphI. In some embodiments, the stability element is at least 90% identical to a sequence less than 750 bp in length and comprising residues 202 to 876 of SEQ ID NO: 1. In other embodiments, the stability element is at least 95% identical to SEQ ID NO: 1.

The invention also provides host cells made by the methods of invention.

The invention further provides recombinant nucleic acid molecules comprising (1) a nucleic acid sequence encoding a nuclease, or a nucleic acid sequence encoding a crRNA activity and a tracrRNA activity that enables site-specific recognition and cleavage of a target site by an RNA-guided DNA endonuclease and (2) a stability element comprising a CEN sequence at least 95% identical to SEQ ID NO: 2 and an ARS consensus sequence at least 90% identical to SEQ ID NO: 3. In some embodiments, the stability element is at least 90% identical to a sequence less than 750 bp in length and comprising residues 202 to 876 of SEQ ID NO: 1. In other embodiments, the stability element is at least 95% identical to SEQ ID NO: 1. The nuclease may be an RNA-guided DNA endonuclease, such as a Cas9) endonuclease. The nuclease may be meganuclease, such as F-CphI.

The invention further provides recombinant nucleic acid molecules and host cells comprising a stability element comprising a CEN sequence and/or ARS consensus sequence shown in SEQ ID NOS: 4, 7, or 10. The invention further provides recombinant nucleic acid molecules and host cells comprising a stability element comprising a sequence that is at least 90% or 95% identical to the CEN sequence and/or ARS consensus sequence shown in SEQ ID NOS: 4, 7, or 10. The invention further provides methods described herein using these recombinant nucleic acid molecules and host cells.

### Definitions

As used in this specification and the appended claims, the singular forms “a.” “an.” and “the” include plural reference unless the context clearly dictates otherwise.

As used herein, the term “stability element” refers to a nucleic acid sequence of between about 200 and about 1300 bp, usually between about 400 and about 900 bp, and often between about 600 and about 750 bp comprising an autonomously replicating sequence (ARS) consensus sequence and, optionally, a centromere sequence (CEN). A stability element allows an extrachromosomal DNA molecule (either linear or circular) comprising the stability element to remain stable in a host cell for extended periods of culturing in non-selective media. The stability elements of the invention typically provide for stability of an extrachromosomal DNA molecule for at least about 10 generations, usually about 20, and often about 30 or more generations in non-selective media.

ARS and CEN sequences have been well studied in yeast. ARSs are origins of DNA replication in yeast chromosomes and are typically short modular DNA sequences comprising an 11-17 bp core sequence element called the ARS Consensus Sequence (ACS), as well as flanking sequences. CEN sequences are part of the complex structures on chromosomes to which spindle fibers attach during meiosis and mitosis. Such sequences are typically between about 100 and about 200 bp long and can be subdivided into three conserved DNA elements CDEI, CDEII and CDEIII. Exemplary CEN sequences of the invention include SEQ ID NOs: 2, 5, 8, and 11. Exemplary ARS consensus sequences of the invention include SEQ ID NOs: 3, 6, 9, and 12).

Exemplary stability elements of the invention are derived from K. marxianus and include SEQ ID NOs: 1, 4, 7, 10, 13, and 14). Also included are subsequences of these sequence which comprise the ARS consensus sequence, optionally a CEN sequence, and any intervening sequences. Exemplary stability elements of this type include residues 202-876 or residues 537-1252 of SEQ ID NO: 1 and residues 1 to 566 or residues 348-1043 of SEQ ID NO: 4.

One of skill will recognize that the exemplified sequences noted above can be modified and still provide stability for extrachromosomal DNA molecules. For example, CEN sequences. ARS consensus sequences, or stability elements having at least about 90%, 91%, 92% 93%, 94%, 95%, 96%, 97%, 98%, or 99% sequence identity to the exemplified sequences are contemplated by the invention. Those of skill in the art readily understand how to determine the identity of two nucleic acids. For example, the identity can be calculated after aligning the two sequences so that the identity is at its highest level. Another way of calculating identity can be performed by published algorithms. For example, optimal alignment of sequences for comparison can be conducted using the algorithm of Needleman and Wunsch, J. Mol. Biol. 48: 443 (1970).

The term “nucleic acid” or “nucleotide” refers to deoxyribonucleic acids (DNA) or ribonucleic acids (RNA) and polymers thereof in either single- or double-stranded form. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); and Rossolini et al., Mol. Cell. Probes 8: 91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA

A “promoter” is defined as one or more a nucleic acid control sequences that direct transcription of a nucleic acid. As used herein, a promoter includes necessary nucleic acid sequences near the start site of transcription. A promoter also optionally includes distal enhancer or repressor elements, which can be located as much as several thousand base pairs from the start site of transcription.

As used herein, the term “marker-less” refers to integration of a donor DNA into a target site within a host cell genome without accompanying integration of a selectable marker. In some embodiments, the term also refers to the recovery of such a host cell without utilizing a selection scheme that relies on integration of selectable marker into the host cell genome. For example, in certain embodiments, a selection marker that is episomal or extrachromasomal may be used to select for cells comprising a plasmid comprising a gRNA. Such use is considered marker-less, as long as the selectable marker is not integrated into the host cell genome.

As used herein, the term “operably linked” refers to a functional linkage between nucleic acid sequences such that the sequences encode a desired function. For example, a coding sequence for a gene of interest, e.g., a selectable marker, is in operable linkage with its promoter and/or regulatory sequences when the linked promoter and/or regulatory region functionally controls expression of the coding sequence. It also refers to the linkage between coding sequences such that they may be controlled by the same linked promoter and/or regulatory region; such linkage between coding sequences may also be referred to as being linked in frame or in the same coding frame. “Operably linked” also refers to a linkage of functional but non-coding sequences, such as an autonomous propagation sequence or origin of replication. Such sequences are in operable linkage when they are able to perform their normal function, e.g., enabling the replication, propagation, and/or segregation of a vector bearing the sequence in a host cell.

As used herein, the term “transformation” refers to a genetic alteration of a host cell resulting from the introduction of exogenous genetic material into the host cell.

As used herein, the term “selecting a host cell expressing a selectable marker” also encompasses enriching for host cells expressing a selectable marker from a population of transformed cells.

As used herein, the term “selectable marker” refers to a gene which functions as guidance for selecting a host cell comprising a marker, for example, a marker expressed by a circular, extrachromosomal nucleic acid in the host cell, as described herein. The selectable markers may include, but are not limited to: fluorescent markers, luminescent markers and drug selectable markers, and the like. The fluorescent markers may include, but are not limited to, genes encoding fluorescence proteins such as green fluorescent protein (GFP), cyan fluorescent protein (CFP), yellow fluorescent protein (YFP), red fluorescent protein (dsRFP) and the like. The luminescent markers may include, but are not limited to, genes encoding luminescent proteins such as luciferases. Drug selectable markers suitable for use with the methods and compositions provided herein include, but are not limited to, resistance genes to antibiotics, such as ampicillin, streptomycin, gentamicin, kanamycin, hygromycin, tetracycline, chloramphenicol, and neomycin. In some embodiments, the selection may be positive selection; that is, the cells expressing the marker are isolated from a population, e.g. to create an enriched population of cells comprising the selectable marker. In other instances, the selection may be negative selection; that is, the population is isolated away from the cells, e.g. to create an enriched population of cells that do not comprise the selectable marker. Separation can be by any convenient separation technique appropriate for the selectable marker used. For example, if a fluorescent marker is used, cells can be separated by fluorescence activated cell sorting, whereas if a cell surface marker has been inserted, cells can be separated from the heterogeneous population by affinity separation techniques, e.g. magnetic separation, affinity chromatography. “panning” with an affinity reagent attached to a solid matrix, or other convenient technique.

“Polypeptide.” “peptide.” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the term “complementary” or “complementarity” refers to specific base pairing between nucleotides or nucleic acids. In some embodiments, for example, and not to be limiting, base pairing between a gRNA (gRNA) and a target site or region in the genome of a host cell is described. Complementary nucleotides are, generally. A and T (or A and U), and G and C. The gRNAs described herein can comprise sequences, for example, a DNA targeting sequence that is perfectly complementary or substantially complementary (e.g., having 1-4 mismatches) to a genomic sequence in a host cell.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-guided DNA endonuclease, Cas9, in complex with a gRNA to recognize and cleave foreign nucleic acid.

As used herein, the terms “cleave,” “cleavage” and/or “cleaving” with respect to homing endonuclease, zinc-finger nuclease, TAL-effector nuclease, or an RNA-guided endonuclease, for example, Cas9, refers to the act of creating a break in a particular nucleic acid. The break can leave a blunt end or sticky end (i.e., 5′ or 3′ overhang), as understood by those of skill in the art. The terms also encompass single strand DNA breaks (“nicks”) and double strand DNA breaks.

As used herein, the term “Cas9” refers to an RNA-guided nuclease (e.g., of bacterial or archeal origin, or derived therefrom). RNA-guided nucleases include the foregoing Cas9 proteins and homologs thereof, and include but are not limited to, Cpf1 (See, e.g., Zetsche et al., Cell, Volume 163, Issue 3, p759-771, 22 Oct. 2015).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1: 10(5): 726-737; Nat. Rev. Microbiol. 2011 June; 9(6): 467-477; Hou, et al., Proc Natl Acad Sci U S A. 2013 Sep. 24; 110(39): 15644-9; Sampson et al., Nature. 2013 May 9; 497(7448): 254-7; and Jinek, et al., Science. 2012 Aug. 17; 337(6096): 816-21. Variants of any of the Cas9 nucleases provided herein can be optimized for efficient activity or enhanced stability in the host cell. Thus, engineered Cas9 nucleases, for example, codon optimized Cas9 nucleases for expression in Kluyveromyces are also contemplated.

As used herein, the terms “modifying,” or “modification,” in the context of modifying a target site in a host cell genome refers to inducing a nucleic acid break in the target site. A modification can be used to edit the genome. As used herein the term “editing” refers to a structural change in the sequence of the genome at a target site. For example, the host cell genome may be edited by deleting or inserting a nucleotide sequence into the genome of the cell. The nucleotide sequence can encode a polypeptide or a fragment thereof. Such editing can be performed, for example, by inducing a double stranded break within a target site in the genome of a host cell, or a pair of single stranded nicks on opposite strands and flanking the target site in the genome of a host cell. Methods for inducing single or double stranded breaks at or within a target site include the use of nucleases, such as a meganuclease, an RNA-guided DNA endonuclease, or a derivatives thereof.

As used herein, the phrases “introducing” or “contacting” in the context of introducing a nucleic acid or protein into a host cell refers to any process that results in the presence of a heterologous nucleic acid or polypeptide inside the host cell. For example, the terms encompass introducing a nucleic acid molecule (e.g., a plasmid or a linear nucleic acid) that encodes the nucleic acid of interest (e.g., an RNA molecule) or polypeptide of interest and results in the transcription of the RNA molecules and translation of the polypeptides. The terms also encompass integrating the nucleic acid encoding the RNA molecules or polypeptides into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein the phrase “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

As used herein the term “homologous recombination” refers to a cellular process in which nucleotide sequences are exchanged between two sufficiently identical molecules of DNA. Two DNA molecules have “sufficient” sequence identity if the two sequences have at least 70%, at least 75%>, at least 80%>, at least 85%>, at least 90%>, at least 95%>, at least 99%>, or 100%, identity between recombination regions, over a length of, for example, at least 15 base pairs, at least 20 base pairs, at least 50 base pairs, at least 100 base pairs, at least 250 base pairs, at least 500 base pairs, or more than 500 base pairs. Those of skill in the art readily understand how to determine the identity of two nucleic acids. For example, the identity can be calculated after aligning the two sequences so that the identity is at its highest level.

Another way of calculating identity can be performed by published algorithms. For example. optimal alignment of sequences for comparison can be conducted using the algorithm of Needleman and Wunsch, J. Mol. Biol. 48: 443 (1970). For a discussion of effective lengths of homology between recombination regions, see Hasty et al. (Mol Cell Biol 11: 5586-91 (1991)).

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term homology directed repair (HDR) refers to a cellular process in which cut or nicked ends of a DNA strand are repaired by polymerization from a homologous template nucleic acid, for example a donor DNA molecule. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid, for example, a donor DNA molecule can be introduced to obtain a specific HDR-induced change of the sequence at the target site. In this way, specific sequences can be introduced at the cut site.

As used herein, the term “KU70)” is used interchangeably with “YKu70)”, and the term “KU80)” is used interchangeably with “YKu80”. The proteins encoded from YKu70) (or KU70) and YKu80) (or KU80) loci are involved in non-homologous end joining. KU exists as a heterodimer of two polypeptides of approximately 70 kDa (generally referred to as YKu70) or KU70 depending organisms), and 80 kDa (generally referred to as YKu80) or KU80) depending on organisms).

### 6. BRIEF DESCRIPTION OF THE FIGURES

FIG. 1 illustrates a schematic diagram of petri dishes showing growth of colonies. FIG. 1 illustrates that the new K. marxianus CEN/ARS plasmid generated is more stable than the same plasmid with a pKD1 element instead of K. marxianus-specific CEN/ARS sequences. Colonies transformed with the K. marxianus CEN/ARS plasmid are larger and rounder than the old colonies, which were rough due to colony sectoring and loss of the plasmid. In addition, colonies containing the new plasmid grew overnight in liquid culture under Nat selection (unlike colonies containing the pKD1 element plasmid), and transformable plasmid was recovered from yeast minipreps.

FIG. 2 illustrates a schematic diagram of petri dishes showing growth of colonies. FIG. 2 illustrates that deletion of YKU70 eliminates NHEJ-mediated circularization of linear plasmids, and homologous recombination is demonstrated by plasmid gap repair in the same background. Wild-type K. marxianus (left) circularizes a linear vector fragment with or without an overlapping gap repair fragment. A YKU70 deleted strain (right) only circularizes the plasmid when the gap repair fragment is supplied.

FIG. 3A and 3B shows transformation of K. marxianus wild-type strains with plasmids containing different K. marxianus CEN/ARS elements. 100 ng of miniprepped plasmid was used in each transformation. FIG. 3A. Transformation of Strain 1; FIG. 3B. Transformation of Strain 2.

FIG. 4 shows the experiment lay-out for K. marxianus transformation using gRNA vector containing different CEN/ARS elements. Both strains served as host for transformation. Each transformation contained 50 ng of linear gRNA vector with CEN/ARS_1 to CEN/ARS_4; 200 ng of gRNA and 500 ng of donor DNA was added to the transformation according to the plate lay-out.

FIG. 5 shows K. marxianus transformation using gRNA vector containing CEN/ARS_5. Both strains served as host for transformation. Each transformation contained 50 ng of linear gRNA vector with CEN/ARS_5; 200 ng of gRNA and 500 ng of donor DNA was added to the transformation according to the plate lay-out.

FIG. 6A-E is a summary of integration at three loci GAS2, NDT80 and GAL80 from transformation of K. marxianus Strain 1. Each locus was screened by colony PCR using sequence-specific primers. 12 colonies were tested for single and double integrations; 30 colonies were tested for triple integrations. GAS2: S; NDT80: N; GAL80: L. FIG. 6A-6E: summary of efficiency of CEN/ARS_1 to CEN/ARS_5.

FIG. 7A-E is a summary of integration at three loci GAS2, NDT80 and GAL80 from transformation of K. marxianus Strain 2. Each locus was screened by colony PCR using sequence-specific primers. 12 colonies were tested for single and double integrations; 30 colonies were tested for triple integrations. GAS2: S; NDT80: N; GAL80: L. FIG. 7A to 7E: summary of efficiency of CEN/ARS_1 to CEN/ARS_5.

### 7. DETAILED DESCRIPTION OF THE EMBODIMENTS

The present invention provides methods of modifying one or more target sites in a Kluyveromyces host cell genome. The methods of the invention use DNA molecules comprising a stability element of the invention that allows the DNA molecules to remain stable in the host cell for multiple generations.

### 7.1 Gap Repair

In some embodiments, modification of the target sites comprises methods which use CRISPR/Cas systems and in vivo assembly of marker and/or gRNA vectors via gap repair, as described in WO2015/095804, which is incorporated herein by reference.

In these methods, the Kluyveromyces host cell, which has reduced non-homologous end joining (NHEJ) activity, is contacted with a linear nucleic acid comprising a stability element of the invention. The linear nucleic acid molecule is capable of homologous recombination with itself or with one or more additional linear nucleic acids contacted with the host cell, whereby homologous recombination in the host cell results in formation of a circular extrachromosomal nucleic acid comprising a coding sequence for a selectable marker and the stability element. The cell also comprises a nuclease capable of cleaving the target site and, optionally, a donor DNA molecule capable of homologous recombination at the cleaved target site, whereby homologous recombination in the host cell results in integration of the donor linear nucleic acid at the target site. Transformed cells are identified by the presence of the selectable marker on the circular extrachromosomal nucleic acid.

The donor DNA molecule is typically heterologous to the host cell and is flanked by nucleotide sequences that are homologous to genomic sequences flanking the target site. In some embodiments, the donor DNA molecule comprises a homologous sequence at the 5′ terminus that is about 70%, 75%, 80%, 85%, 90%, 95% or 100% homologous to a 5′ region of a selected genomic target site In some embodiments, the donor DNA molecule comprises a homologous sequence at the 3′ terminus that is about 70%, 75%, 80%, 85%, 90%, 95% or 100% homologous to a 3′ region of a selected genomic target site. In some cases, each of the homologous sequences flanking the donor DNA molecule comprises from about 50 to about 1500 nucleotides.

The donor DNA molecule may comprise any nucleic acid of interest. For example, the donor DNA molecule may comprise a gene of interest that can be knocked in to a host genome. In other embodiments, the donor DNA molecule functions as a knockout construct that is capable of specifically disrupting a target gene upon integration of the construct into the target site of the host cell genome, thereby rendering the disrupted gene non-functional. Examples of nucleic acids of interest include, but are not limited to, a protein-coding sequence, a promoter, an enhancer, terminator, transcriptional activator, transcriptional repressor, transcriptional activator binding site, transcriptional repressor binding site, intron, exon, poly-A tail, multiple cloning site, nuclear localization signal, mRNA stabilization signal, integration loci, epitope tag coding sequence, degradation signal, or any other naturally occurring or synthetic DNA molecule. In specific embodiments, the nucleic acid of interest does not comprise a nucleic acid encoding a selectable marker.

NHEJ activity in the host cell may be disrupted in a number of ways. Typically, a gene locus that is involved in NHEJ activity of the cell is disrupted. For example, the YKU70) gene locus may be disrupted, such that NHEJ activity is reduced in the cell. In some cases, the YKU70) gene locus is disrupted by inserting or integrating a nucleic acid encoding an RNA-guided endonuclease in the YKU70 gene locus. The reduction in NHEJ activity can be a reduction of at least 20%, 30%, 40%, 50%, 60%, 70%, 80%, 90%, 100% or any percent reduction in between these percentages, as compared to a Kluyveromyces cell that does not have a disruption in a gene controlling NHEJ in the cell.

In some embodiments, the RNA-guided DNA endonuclease is provided by introducing a nucleic acid encoding the endonuclease into the host cell. For example, a plasmid or vector comprising a stability element of the invention and a nucleic acid encoding the RNA-guided DNA endonuclease can be introduced into the cell. In some embodiments, the plasmid can further comprise a nucleic acid sequence encoding a selectable marker for maintenance of the plasmid in the host cell. In some embodiments the nucleic acid encoding the endonuclease further comprises a promoter sequence. In some embodiments, the nucleic acid encoding the RNA-guided DNA endonuclease is integrated into genome of the host cell. In certain embodiments, the RNA-guided DNA endonuclease, for example, Cas9), is integrated into the YKU70 gene of the Kluyveromyces host cell, thereby reducing NHEJ activity in the yeast cell. In some embodiments, the nucleic acid encoding the RNA-guided DNA endonuclease is under the control of a constitutive promoter. In some embodiments, the RNA-guided DNA endonuclease can be introduced into the host cell prior to, simultaneously with, or after introduction of the first and second linear nucleic acids. In other embodiments, the RNA-guided DNA endonuclease can be introduced into the host cell prior to, simultaneously with, or after introduction of the first linear nucleic acids, the second linear nucleic acid and the donor DNA molecule.

In some embodiments, the first linear nucleic acid comprises two internal homologous sequences that are capable of homologously recombining with each other, whereby homologous recombination of the internal homologous sequences results in formation of the circular extrachromosomal nucleic acid comprising a stability element of the invention and expressing the selectable marker. Once circularized, the extrachromosomal nucleic acid includes a coding sequence for a selectable marker, and suitable regulatory sequences such as a promoter and/or a terminator that enables expression of the marker in the host cell. Providing the selectable marker on a circular, extrachromosomal nucleic acid, allows markerless integration of one or more donor DNA molecules into a host cell genome, while avoiding the integration of extraneous sequences (i.e., a selectable marker) into the genome and any deleterious effects associated with prolonged marker expression.

In some embodiments, the methods of the invention provide for markerless recovery of a transformed host cell comprising a successfully integrated donor nucleic acid. Such a cell occurs within a frequency of about one every 1000, 900, 800, 700, 600, 500, 400, 300, 200 or 100 contacted host cells, or clonal populations thereof, screened. In particular embodiments, markerless recovery of a transformed host cell comprising a successfully integrated donor nucleic acid occurs within a frequency of about one every 90, 80, 70, 60, 50, 40, 30, 20, or 10 contacted host cells, or clonal populations thereof, screened. In more particular embodiments, markerless recovery of a transformed cell comprising a successfully integrated donor nucleic acid occurs within a frequency of about one every 9, 8, 7, 6, 5, 4, 3, or 2 contacted host cells, or clonal populations thereof, screened.

A variety of methods are available to identify those cells having an altered genome at or near the target site without the use of a selectable marker. In some embodiments, such methods seek to detect any change in the target site, and include but are not limited to PCR methods, sequencing methods, nuclease digestion, e.g., restriction mapping. Southern blots, and any combination thereof. Phenotypic readouts, for example, a predicted gain or loss of function, can also be used as a proxy for effecting the intended genomic modification(s).

In some embodiments, the first linear nucleic acid comprising a selectable marker is capable of recombining with a second linear nucleic acid encoding, for example, one or more gRNAs. After introduction of the first and second linear nucleic acids, the first and second linear nucleic acids undergo homologous recombination to form a circular, episomal or extrachromosomal nucleic acid comprising the coding sequence for the selectable marker and the one or more gRNAs.

Subsequent to formation of the extrachromosomal nucleic acid comprising the coding sequence for the selectable marker and the gRNA, the gRNA is transcribed from the extrachromosomal nucleic acid and guides the RNA-guided DNA endonuclease expressed in the host cell to a target site in the genome of the host cell, where the endonuclease creates a break at the target site.

In typical embodiments, the methods of the invention are used to integrate a plurality (i.e., two or more) donor DNA molecules into a plurality of target sites of the host cell genome. In these embodiments, the Kluyveromyces host cell is contacted with a first linear nucleic acid and two or more second linear nucleic acid molecules, wherein each second linear nucleic acid molecule comprises a nucleic acid encoding a different gRNA which targets a different site in the host cell genome. Each different second linear nucleic acid can recombine with the first linear nucleic acid to form two or more different, circular, extrachromosomal nucleic acids in the host cell. It is understood that the term “first linear nucleic acid” and “second linear nucleic acid” includes multiple copies of the same nucleic acid molecule. For example, the host cell can be contacted with two or more second linear nucleic acid molecules, wherein each second linear nucleic acid molecule comprises a nucleic acid encoding a different gRNA to target two, three, four, five, six, seven or more different sites in the host cell genome. In some embodiments, once the gRNA guides the RNA-guided endonuclease to two or more target sites, the endonuclease creates a break at the two or more target sites and two or more donor DNA molecules are integrated into the host cell genome via homologous recombination.

In some embodiments, the first linear nucleic acid comprising a selectable marker is a gapped vector comprising a pair of homologous flanking sequences that recombine with a pair of homologous sequences flanking the gRNA cassette in the second linear nucleic acid to form a larger circular vector where the gap has been repaired by inserting the second linear nucleic acid into the gapped vector. In some embodiments each homologous flanking sequence of the pair of homologous flanking sequences in the first nucleic acid contains a recombination region comprising a nucleotide sequence of sufficient length and sequence identity that allows for homologous recombination with the pair of homologous flanking sequences in the second linear nucleic acid, but not with other regions of the first or second linear nucleic acid participating in the in vivo assembly, nor with any genomic regions of the host cell. For in vivo assembly of marker/gRNA vectors via gap repair and for selection of cells capable of homologous recombination and gap repair, see, for example, Horwitz et al. (Cell Systems 1: 88-96 (2015)) and WO2015/095804, both of which are incorporated herein in their entireties by this reference.

In some embodiments, the gRNA is introduced into the cell on circular extrachromosomal nucleic acid (i.e., a plasmid) that is not formed through homologous recombination of linear nucleic acid molecules. In these embodiments, the plasmid comprises a stability element of the invention. These embodiments are used, for example, when integration of a single donor DNA molecule is desired.

Using the methods provided herein, one or more target sites in a host cell genome can be modified with surprisingly high efficiency compared to conventional CRISPR/Cas systems. The efficiency of alteration in a population of cells can be at least about 5%, 10%, 15%, 20%, 25%, 30%, 35%, 40%, 45%, 50%, 55%, 60%, 65%, 70%, 75% or 80% or higher, or any percentage in between these percentages.

As used throughout, a guide RNA (gRNA) sequence is a sequence that interacts with an RNA-guided DNA endonuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Each gRNA contains a gRNA scaffold sequence that binds to the RNA-guided DNA endonuclease that does not comprise the DNA targeting sequence. In some embodiments, the gRNA comprises a crRNA sequence and a transactivating crRNA (tracrRNA) sequence. In some embodiments, the gRNA does not comprise a tracrRNA sequence.

Generally, the DNA targeting sequence is designed to complement (e.g., perfectly complement) or substantially complement the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%).

Any RNA-guided DNA endonuclease can be used in the methods provided herein. In some embodiments, the RNA-guided DNA endonuclease is an active Cas9 endonuclease such that when bound to a target nucleic acid as part of a complex with a gRNA, a double strand break is introduced into the target nucleic acid. In some embodiments, the double strand break is repaired by HDR to insert a donor DNA molecule into the genome of the host cell. Various Cas9 endonucleases can be used in the methods described herein. For example, a Cas9 nuclease that requires an NGG protospacer adjacent motif (PAM) immediately 3′ of the region targeted by the gRNA can be utilized. As another example, Cas9 proteins with orthogonal PAM motif requirements can be used to target sequences that do not have an adjacent NGG PAM sequence. Exemplary Cas9 proteins with orthogonal PAM sequence specificities include, but are not limited to, those described in Esvelt et al. (Nature Methods 10: 1116-1121 (2013)).

In some cases, the Cas9 protein is a nickase, such that when bound to target nucleic acid as part of a complex with a gRNA, a single strand break or nick is introduced into the target nucleic acid. A pair of Cas9 nickases, each bound to a different gRNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation (See, for example, Ran et al. “Double nicking by RNA-guided CRISPR Cas9 for enhanced genome editing specificity,” Cell 154(6): 1380-1389 (2013)).

### 7.2 Site-Specific Nucleases

In some embodiments, modification of the target sites comprises methods which use extrachromosomal DNA molecules, which comprise a stability element of the invention and one or more nucleic acid sequence encoding a nuclease, as described in WO 2012/149470, which is incorporated herein by reference.

In these methods, a donor DNA molecule is introduced into a Kluyveromyces host cell, wherein the donor DNA comprises a nucleic acid of interest flanked by a first homology region and a second homology region. The first and second homology regions share homology with 5′ and 3′ regions, respectively, of the genomic target site. An extrachromosomal DNA comprising a stability element of the invention and a nucleic acid sequence encoding site-specific nuclease is also introduced to the host cell. The nuclease is capable of recognizing and cleaving a unique recognition sequence (also called a landing pad) within the target site. Upon induction of a double-stranded break within the target site by the site-specific nuclease, endogenous homologous recombination machinery integrates the nucleic acid of interest at the cleaved target site at a higher frequency as compared to a target site not comprising a double-stranded break. This increased frequency of integration obviates the need to co-integrate a selectable marker in order to select transformants having undergone a recombination event.

A variety of methods are available to identify those cells having an altered genome at or near the target site without the use of a selectable marker. In some embodiments, such methods seek to detect any change in the target site, and include but are not limited to PCR methods, sequencing methods, nuclease digestion, e.g., restriction mapping, Southern blots, and any combination thereof.

The methods of the invention can be used for simultaneous genomic integration of a plurality of exogenous nucleic acids of interest using a plurality of site-specific nucleases. These methods, for example, allow for the simultaneous integration of a plurality of genes in a single enzymatic pathway.

As in the case for gap repair embodiments described above, the donor DNA molecule may comprise any nucleic acid of interest. For example, the donor DNA molecule may comprise a gene of interest that can be knocked in to a host genome. In other embodiments, the donor DNA molecule functions as a knockout construct that is capable of specifically disrupting a target gene upon integration of the construct into the target site of the host cell genome, thereby rendering the disrupted gene non-functional. Examples of nucleic acids of interest include, but are not limited to, a protein-coding sequence, a promoter, an enhancer, terminator, transcriptional activator, transcriptional repressor, transcriptional activator binding site, transcriptional repressor binding site, intron, exon, poly-A tail, multiple cloning site, nuclear localization signal, mRNA stabilization signal, integration loci, epitope tag coding sequence, degradation signal, or any other naturally occurring or synthetic DNA molecule. In specific embodiments, the nucleic acid of interest does not comprise a nucleic acid encoding a selectable marker.

As noted above, a double-strand break at a selected target site is induced by site specific endonucleases, for example, site-specific recombinases, transposases, topoisomerases. and zinc finger nucleases, and include modified derivatives, variants, and fragments thereof. The nuclease cleaves the target site at a recognition sequence, that is specifically recognized and/or bound by a double-strand break inducing agent. The length of the recognition sequence can vary, and includes, for example, sequences that are at least 10, 12, 14, 16, 18, 19, 20, 21. 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length.

In some embodiments, the recognition sequence is palindromic, that is, the sequence on one strand reads the same in the opposite direction on the complementary strand. In some embodiments, the nick/cleavage site is within the recognition sequence. In other embodiments, the nick/cleavage site is outside of the recognition sequence. In some embodiments, cleavage produces blunt end termini. In other embodiments, cleavage produces single-stranded overhangs, i.e., “sticky ends,” which can be either 5′ overhangs, or 3′ overhangs.

The recognition sequence within the selected target site can be endogenous or exogenous to the host cell genome. When the recognition site is exogenous to the host cell genome, it may be introduced into the host cell genome by any means known to those of skill in the art. For example, the recognition sequence can be introduced using the gap-repair methods described above. The recognition sequence is typically recognized by a naturally-occurring double-strand break inducing agent. Alternatively, a recognition site could be recognized and/or bound by a modified or engineered double-strand break inducing agent designed or selected to specifically recognize the recognition sequence to produce a double-strand break. In some embodiments, the modified double-strand break inducing agent is derived from a native, naturally-occurring double-strand break inducing agent. In other embodiments, the modified double-strand break inducing agent is artificially created or synthesized. Methods for selecting such modified or engineered double-strand break inducing agents are known in the art. For example, amino acid sequence variants of the protein(s) can be prepared by mutations in the DNA. Methods for mutagenesis and nucleotide sequence alterations include, for example, Kunkel, (1985) Proc Natl Acad Sci USA 82: 488-92; Kunkel, et al., (1987) Meth Enzymol 154: 367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff, et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double strand break inducing activity are known and generally measure the overall activity and specificity of the agent on DNA substrates containing recognition sites.

Endonucleases useful in the present invention include homing endonucleases. which like restriction endonucleases, bind and cut at a specific recognition sequence. However the recognition sites for homing endonucleases are typically longer, for example, about 18 bp or more. Homing endonucleases. also known as meganucleases, are well known to those of skill in the art and have been classified into the following families based on conserved sequence motifs: an LAGLIDADG homing endonuclease, an HNH homing endonuclease, a His-Cys box homing endonuclease, a GIY-YIG homing endonuclease, and a cyanobacterial homing endonuclease. Examples of homing endonuclease useful in the present invention include, but are not limited to: H-Drel. I-Seel, I-Scell, I-SceIII, I-SceIV, I-SceV, I-See VI, ISceVII, I-Ceul, I-CeuAIIP, I-Crel, I-CrepsbIP, I-CrepsbIIP, I-CrepsbIIIP, I-CrepsbIVP, I-Tlil, I-Ppol, Pi-Pspl, F-Scel, F-SceII, F-Suvl, F-Cphl, F-Tevl, F-TevII, I-Amal, I-Anil, I-Chul, ICmoel, I-CpaI, I-CpaII, I-Csml, I-Cvul, I-CvuAIP, I-Ddil, I-DdiII, I-Dirl, I-Dmol, I-Hmul, IHmuII, I-HsNIP, I-LlaI, I-Msol, I-Naal, I-Nanl, I-NclIP, I-NgrIP, I-NitI, I-NjaI, I-Nsp236IP, IPakl, I-PboIP, I-PcuIP, I-PcuAI, I-PcuVI, I-PgrIP, I-PobIP, I-Porl, I-PorIIP, I-PbpIP, ISpBetaIP, I-Seal, I-SexIP, I-SneIP, I-Spoml, I-SpomCP, I-SpomIP, I-SpomIIP, I-SquIP, ISsp68031, I-SthPhiJP, I-SthPhiST3P, I-SthPhiSTe3bP, I-TdeIP, I-Tevl, I-TevII, I-TevIII, IUarAP, I-UarHGPAIP, I-UarHGPA13P, I-VinIP, I-ZbiIP, PI-MgaI, PI-Mtul, PI-MtuHIP PIMtuHIIP, PI-PfuI, PI-PfuII, PI-PkoI, PI-PkoII, PI-Rma43812IP, PI-SpBetaIP, PI-Seel, PI-Tful, PI-TfuII, PI-ThyI, PI-Tlil, or PI-TliII, or any variant or derivative thereof.

In some embodiments the nuclease is a TAL-effector DNA binding domain-nuclease fusion protein (TALEN). TAL effectors of plant pathogenic bacteria in the genus Xanthomonas play important roles in disease, or trigger defense, by binding host DNA and activating effector-specific host genes. The TAL-effector DNA binding domain may be engineered to bind to a desired target sequence, and fused to a nuclease domain, e.g., from a type II restriction endonuclease, typically a nonspecific cleavage domain from a type II restriction endonuclease such as FokI, HhaI, HindIII, Nod, BbvCI, EcoRI, BglI, and AlwI. Thus, in preferred embodiments, the TALEN comprises a TAL effector domain comprising a plurality of TAL effector repeat sequences that, in combination, bind to a specific nucleotide sequence in the target DNA sequence, such that the TALEN cleaves the target DNA within or adjacent to the specific nucleotide sequence. TALENS useful for the methods provided herein include those described in WO10/079430 and U.S. Patent Application Publication No. 2011/0145940.

In some embodiments the nuclease is a site-specific recombinase. A site-specific recombinase, also referred to as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. In some embodiments, the recombinase is a serine recombinase or a tyrosine recombinase. In some embodiments, the recombinase is from the Integrase or Resolvase families. In some embodiments, the recombinase is an integrase selected from the group consisting of FLP, Cre, lambda integrase, and R.

In some embodiments the nuclease is a transposase. Transposases are polypeptides that mediate transposition of a transposon from one location in the genome to another. Transposases typically induce double strand breaks to excise the transposon, recognize subterminal repeats, and bring together the ends of the excised transposon, in some systems other proteins are also required to bring together the ends during transposition. Examples of transposons and transposases include, but are not limited to, the Ac/Ds, Dt/rdt, Mu-Ml/Mn, and Spm(En)/dSpm elements from maize, the Tam elements from snapdragon, the Mu transposon from bacteriophage, bacterial transposons (Tn) and insertion sequences (IS), Ty elements of yeast (retrotransposon), Ta 1 elements from Arabidopsis (retrotransposon), the P element transposon from Drosophila, the Copia, Mariner and Minos elements from Drosophila, the Hermes elements from the housefly, the Piggy Back elements from Trichplusia ni, Tc1 elements from C. elegans, and IAP elements from mice (retrotransposon).

In some embodiments the nuclease is a zinc-finger nuclease (ZFN). ZFNs are engineered double-strand break inducing agents comprised of a zinc finger DNA binding domain and a double strand break inducing agent domain. Engineered ZFNs consist of two zinc finger arrays (ZFAs), each of which is fused to a single subunit of a non-specific endonuclease, such as the nuclease domain from the FokI enzyme, which becomes active upon dimerization.

### 7.3 Cell Culture

The Kluyveromyces host cells are cultured using methods well known to those of skill in the art. If a selectable maker is used, the cells are cultured for a period of time sufficient for expression of the selectable marker from the circularized extrachromosomal vector. In some embodiments where the selectable marker is a drug resistance marker, the culturing is carried out for a period of time sufficient to produce an amount of the marker protein that can support the survival of cells expressing the marker in selectable media. In certain embodiments, these conditions also select against the survival of cells not expressing the selectable marker. Selective pressure can be applied to cells using a variety of compounds or treatments that would be known to one of skill in the art. For example, selective pressure can be applied by exposing host cells to conditions that are suboptimal for or deleterious to growth, progression of the cell cycle or viability, such that cells that are tolerant or resistant to these conditions are selected for compared to cells that are not tolerant or resistant to these conditions. Conditions that can be used to exert or apply selective pressure include, but are not limited to, antibiotics, drugs, mutagens, compounds that slow or halt cell growth or the synthesis of biological building blocks, compounds that disrupt RNA, DNA or protein synthesis, deprivation or limitation of nutrients, amino acids, carbohydrates or compounds required for cell growth and viability from cell growth or culture media, treatments such as growth or maintenance of cells under conditions that are suboptimal for cell growth, for instance at suboptimal temperatures, atmospheric conditions (e.g., % carbon dioxide, oxygen or nitrogen or humidity) or in deprived media conditions. The level of selective pressure that is used can be determined by one of skill in the art. This can be done, for example, by performing a kill curve experiment, where control cells and cells that comprise resistance markers or genes are tested with increasing levels, doses, concentrations or treatments of the selective pressure and the ranges that selected against the negative cells only or preferentially over a desired range of time (e.g., from 1 to 24 hours, 1 to 3 days, 3 to 5 days, 4 to 7 days, 5 to 14 days, 1 to 3 weeks, 2 to 6 weeks). The exact levels, concentrations, doses, or treatments of selective pressure that can be used depends on the cells that are used, the desired properties themselves, the markers, factors or genes that confer resistance or tolerance to the selective pressure as well as the levels of the desired properties that are desired in the cells that are selected and one of skill in the art would readily appreciate how to determine appropriate ranges based on these considerations.

The culturing can be performed in a suitable culture medium in a suitable container, including but not limited to a cell culture plate, a flask, or a fermentor. In some embodiments, the culture medium is an aqueous medium comprising assimilable carbon, nitrogen and phosphate sources. Such a medium can also include appropriate salts, minerals, metals and other nutrients. In some embodiments, in addition to the selection agent, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter). Materials and methods for the maintenance and growth of cell cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process. In some embodiments, the culturing is carried out for a period of time sufficient for the transformed population to undergo a plurality of doublings until a desired cell density is reached. In some embodiments, the culturing is carried out for a period of time sufficient for the host cell population to reach a cell density (OD600) of between 0.01 and 400 in the fermentation vessel or container in which the culturing is being carried out. In other embodiments, the culturing is carried for a period of at least 12, 24, 36, 48, 60, 72, 84, 96 or more than 96 hours. In some embodiments, the culturing is carried out for a period of between 3 and 20 days. In some embodiments, the culturing is carried out for a period of 1, 2, 3, 4, 5, 6, 7, 8, 9, 10, 11, 12, 13, 14, 15, 16, 17, 18, 19, 20 or more than 20 days.

In some embodiments of the methods described herein, the methods further comprise the step of eliminating the circularized extrachromosomal vector from the host cell. for example, once a selected host cell has been identified as comprising the desired genomic integration(s). Plasmid-based systems generally require selective pressure on the plasmids to maintain the foreign DNA in the cell. In some embodiments, elimination of a plasmid encoding the selective marker from a selected cell can be achieved by allowing the selected cells to undergo sufficient mitotic divisions such that the plasmid is effectively diluted from the population. Alternatively, plasmid-free cells can be selected by selecting for the absence of the plasmid, e.g., by selecting against a counter-selectable marker (such as, for example, URA3) or by plating identical colonies on both selective media and non-selective media and then selecting a colony that does not grow on the selective media but does grow on the non-selective media.

### 7.4 Host Cells

The methods of the invention can be used to modify one or more target sites in a Kluyveromyces host cell genome. The host cell can be any member of the genus Kluyveromyces, including, for example, K. marxianus, K lactis, K. aestuarii K. africanus, K. bacillisporus K. blattae, K. dobzhanskii, K. hubeiensis, K. lodderae, K. nonfermentans, K. piceae, K. sinensis, K. thermotolerans, K. waltii, K. wickerhamii, and K. yarrowii.

### 7.5 Methods of Producing a Product of Interest

As noted above, the donor DNA can be used integrate any desired nucleic acid sequence into the genome of the Kluyveromyces host cell. Thus, the methods of the invention comprise culturing a host cell comprising one or more integrated donor DNA molecules of interest encoding one or more proteins of interest under conditions suitable for production of the protein and recovering the protein produced by the host cell. Methods for preparing purified proteins from cell cultures are well known to those of skill in the art. In some embodiments, the protein of interest is a protein selected from the group consisting of an antibody, an enzyme, a hormone, a growth factor, an anticoagulant, blood factors, an engineered protein, an interferon, an interleukin, a thrombolytic, a viral protein or a bacterial protein.

In some embodiments, one or more secretion signal sequences (e.g., two, three, four, five, six, seven, eight, nine, or ten secretion signal sequences) may be inserted in the donor DNA molecules. The secretion signal sequence encodes a secretion signal peptide that is recognized by the molecular machinery of the host cell, which then secretes the protein from the cell. The choice of a secretion signal peptide may depend on the type of the host cell.

In some embodiments, the nucleic acid sequence(s) encoding the polypeptide of interest may be codon optimized according to codon frequencies of the host cell. Using the codon with the highest occurrence frequency in the host cell may reduce unwanted mutations and improve translation efficiency. The donor DNA molecules may also include appropriate expression control elements known in the art, including promoters, enhancers, selection markers, and transcription terminators well known to those of skill in the art. Methods for expressing therapeutic proteins are known in the art. See, for example, Paulina Balbas, Argelia Lorence (eds.) Recombinant Gene Expression: Reviews and Protocols (Methods in Molecular Biology), Humana Press; 2nd ed. 2004 edition (Jul. 20, 2004); Vladimir Voynov and Justin A. Caravella (eds.) Therapeutic Proteins: Methods and Protocols (Methods in Molecular Biology) Humana Press; 2nd ed. 2012 edition (Jun. 28, 2012).

The methods and compositions described herein are also useful in introducing multiple modifications in the genome of the host cell and thus provide particular advantages for constructing recombinant organisms comprising optimized biosynthetic pathways, for example, towards the conversion of biomass into biofuels, pharmaceuticals or biomaterials. Functional non-native biological pathways have been successfully constructed in microbial hosts for the production of a number of valuable products, including precursors to the antimalarial drug artemisinin, fatty acid derived fuels and chemicals (e.g., fatty esters, fatty alcohols and waxes), methyl halide derived fuels and chemicals, polyketide synthases that make cholesterol lowering drugs, and polyketides.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### 8. Example 1

### 8.1 Materials and Methods

8.1.1. Preparation of K. marxianus Host Strain for CRISPR-Cas

A wild-type K. marxianus strain was used. A S. cerevisiae codon-optimized version of the Streptococcus pyogenes Cas9 gene was fused to an SV40 nuclear localization sequence designed and cloned into an integration cassette under the expression of the S. cerevisiae TEFI promoter with a CYCI terminator (Horwitz et al., 2015, Cell Systems (1): 88-96; DiCarlo et al. 2013, Nucleic Acids Res 2013 41(7) 4336-43); see sequence listing). The construct, marked with an hphA (hygromycin resistance) cassette, was stably integrated at the YYKU70 locus of wild-type K. marxianus strain. The integration construct has a nucleic acid sequence of SEQ ID NO: 23. Correct integration at YYKU70 locus was verified by colony PCR reactions.

### 8.1.2. Construction of Stable Plasmids for Kluyveromyces

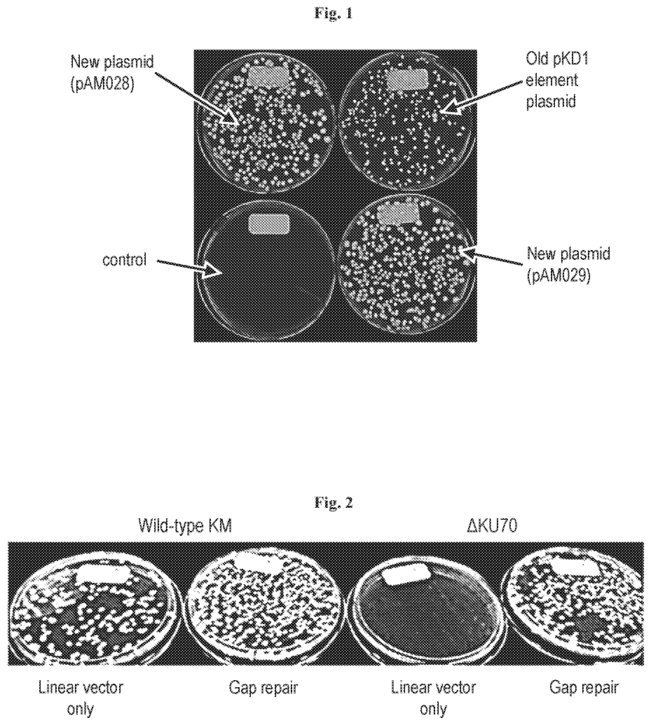

New stable plasmids for replicating and/or expressing a gRNA or nucleases were prepared. The new stable plasmids contain K. marxianus specific stability element comprising a centromere sequence (CEN) and an autonomously replicating consensus sequence (ARS). Plasmid pAM028 contains the CEN/ARS sequences shown in SEQ ID NO: 1. Plasmid pAM029 contains the CEN/ARS sequence shown in SEQ ID NO: 4. The “Old pKD1 element plasmid” shown in FIG. 1 has a pKD1 stability element from a pKD1 plasmid (Chen, Wesolowski-Louvel et al., J Basic Microbiol 28(4): 211-220, 1988) instead of the CEN/ARS sequences shown in SEQ ID NOs: 1 or 4.

Using homologous recombination, the stability element (comprising ARS/CEN sequence shown in SEQ ID NO:1) was cloned into a linearized vector containing a promoter driving RNA expression, terminator, chimeric gRNA sequence, origin of replication, bacterial and yeast antibiotic selection markers. The plasmid is referred to as K. marxianus gRNA entry vector (sequence shown in SEQ ID NO: 21). Once this plasmid was created and verified, it was used to generate a stable plasmid for meganuclease expression (sequence shown in SEQ ID NO: 22). A linear version of this plasmid was generated by PCR with the K. marxianus CEN/ARS, origin of replication, bacterial and yeast antibiotic selection markers. A PCR product containing the f-CphI open reading frame, promoter and terminator was also amplified and cloned into the linear fragment described above.

### 8.1.3. Guide-RNA Expression Cassettes

Cas9 protein is targeted to cut sites by association with a generic structural RNA and a specific targeting RNA. The standard “chimeric” configuration was adopted, in which the targeting and structural RNAs are fused to create a single gRNA. Expression of the gRNA construct was driven by the SNR52 polymerase III promoter, with a SUP4 terminator. The gRNA cassette was cloned into low copy, stable vector using the K. marxianus chromosome V CEN/ARS elements by gap repair directly into a Cas9-expressing host strain (Orr-Weaver et al., 1983). The low copy, stable vector for gRNA entry has a sequence shown in SEQ ID NO: 21. In order to gap repair the gRNA cassette/s directly into the expression vector in the host strain, we first generated full-length gRNA cassettes with 500 bp flanking homology to the linearized vector. In certain embodiments, we co-transformed host cells with single or multiple DNA fragments (for multiplexing) containing gRNA cassettes bearing flanking homology to the linear plasmid.

### 8.1.4. Selection of Target Sites and Generation of Donor DNA

Candidate CRISPR target sites inside the targeted open reading frames (ORFs) were identified based on the presence of a PAM sequence N(19)NGG. The following genomic loci were selected as target sites: NDT80, YKU80, GAS2, GAL80, and LEU2. The gRNA sequences for these target sites are shown as SEQ ID NOs: 15-20. Donor DNA constructs with 500 bp of flanking homology were generated using standardized linkers for assembly as described in U.S. Pat. No. 8,110,360, which is hereby incorporated by reference in its entirety. See also, Horwitz et al. 2015.

8.1.5. K. marxianus Transformation Protocol and Genomic Integrations of Markerless DNA

The K. marxianus transformation protocol we developed was a variant of the Gietz et al. 2007 (Nat Protoc 2: 31-34) adapted for these strains. After 1-2 days growth on agar plates, single colonies were picked and used to inoculate 3ml of liquid rich media (e.g., YPD). This liquid culture was growth overnight with shaking and then diluted into fresh media the next morning to an OD of 0.05-0.2. The diluted culture was grown with shaking incubation until an OD of 0.6-0.9, undergoing a minimum of two doublings. 5 ml of culture was harvested for each transformation, centrifuged to precipitate cells (7000× g, 2 minutes) and then resuspended in an equal volume of sterile water. Centrifugation was repeated, and cells were resuspended in 100 mM lithium acetate. Centrifugation was repeated one last time, and cells were resuspended in 100 mM lithium acetate at a volume 250-fold lower than the original volume (e.g., 20 μl for an original volume of 5 ml). To each transformation, 240 μl of 50% PEG solution, 36 μl of 1.5M lithium acetate, 10 μl of boiled salmon sperm DNA. 54 μl transformation DNA mixed with water and 20 μl cell mixture were added. The transformation mixture was briefly vortexed to distribute cells and reagents evenly, followed by incubation at 30° C. for 30 minutes then 42° for 40 minutes. After heat shock, the transformation mixture was centrifuged again at lower speed (3000× g, 2 minutes), followed by aspiration of the PEG suspension and resuspension of the cell pellet in 2 ml liquid rich media. This culture was then incubated with shaking overnight followed by plating onto selective media. Each marker-less integrations were confirmed using a colony PCR.

### 8.2 Results and Discussion

### 8.2.1. Establishment of High-Efficiency, High Throughput Integrations in Kluyveromyces Using CRISPR-Cas

The molecular biology tools available for Kluyveromyces marxianus (KM) genetic engineering were not as robust as those used in Saccharomyces cerevisiae (SC). Currently. only one report of CRISPR use in K. marxianus for knockouts reliant upon random mutation has been published in the literature. (See Lobs et al. CRISPR-Cas9-enabled genetic disruptions for understanding ethanol and ethyl acetate biosynthesis in Kluyveromyces marxianus. 2017. Biotechnology for Biofuels). A high-throughput method would require higher rates of marker-less knockout/integration if markerless, multiplex engineering in K. marxianus would be achieved. As used herein, the term “markerless” refers to integration of a donor DNA into a target site within a host cell genome without accompanying integration of a selectable marker. In some embodiments, the term also refers to the recovery of such a host cell without utilizing a selection scheme that relies on integration of selectable marker into the host cell genome. For example, in certain embodiments, a selection marker that is episomal or extrachromasomal may be utilized to select for cells comprising a plasmid encoding a nuclease capable of cleaving a genomic target site. Such use would be considered “markerless” so long as the selectable marker is not integrated into the host cell genome.

The first step toward obtaining higher transformation efficiencies was construction of a more stable plasmid. Previously, a plasmid containing a pKD1-element from K. lactis was used in K. marxianus, and transformants were obtained, but the plasmid was unstable (FIG. 1, top right). Colonies transformed with this plasmid showed evidence of colony sectoring, as small colonies with rough edges are formed when the plasmid is lost at high frequency. Two new plasmids pAM028 and pAM029 containing K. marxianus-specific CEN/ARS sequences (SEQ ID NO: 1 and SEQ ID NO: 4, respectively) were made (FIG. 1, top left and bottom right). The CEN/ARS sequences were incorporated to generate stable plasmids. Colonies transformed with these plasmids are large, round, and smooth, indicating stable expression of the antibiotic-resistance cassette. In addition, plasmids can be recovered from these colonies, demonstrating that the new plasmids are maintained and not integrated.

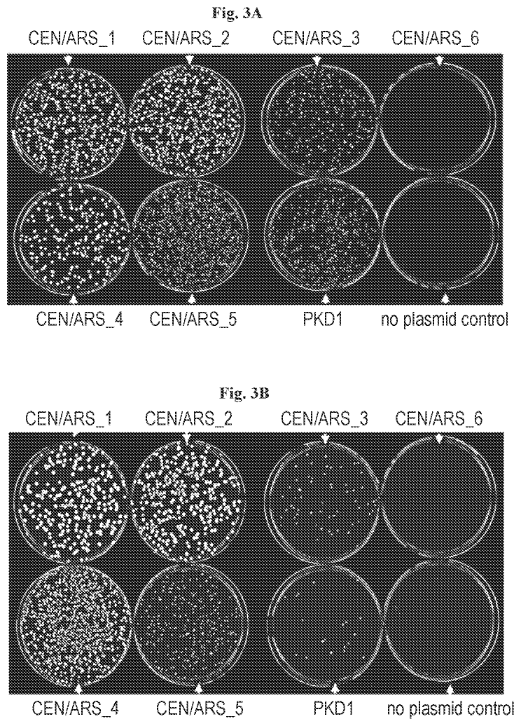

The second step towards high genomic integration efficiency was achieved by reducing or eliminating genomic double-strand break repair via NHEJ. NHEJ relies on the YKU complex (a heterodimer of YKU70) and YKU80 proteins) and DNA ligase. By deleting YKU70, the level of NHEJ was greatly reduced. “Gap repair” of plasmids by homologous recombination was achieved. (see FIG. 2). Plasmid gap repair occurs when multiple overlapping linear fragments generate an intact, selectable circular vector (see, WO2015/095804, which is incorporated herein by reference).

Together, a stable plasmid and elimination of NHEJ led to the first high-efficiency markerless integration in K. marxianus using CRISPR-Cas. In this example, three components were used for efficiently targeted integrations or deletions using CRISPR-Cas: expressed Cas9 protein, gRNA specific for the targeted locus, and donor DNA for repair of the induced double-strand break. A construct for Cas9 expression was previously integrated into a K. marxianus strain, disrupting the YKU70 locus. A chimeric gRNA was expressed from a Nat-marked K. marxianus plasmid using the Saccharomyces cerevisiae pSNR52 promoter and SUP4 terminator. (See Horwitz et al., 2015, Cell Systems, (1)88-96). Donor DNA was supplied as linear MssI-digested fragment with 500bp of GAL80 upstream and downstream homology flanking a GFP expression construct. When Donor DNA was not supplied, no colonies were recovered. When donor DNA was supplied, many colonies grew. (Data not shown). This phenotype is indicative of double-strand breaks induced by gRNA expression at a single location (GAL80) repaired by homologous recombination with the supplied donor DNA construct. cPCR of 24 colonies of marker-less integration at GAL80 show that 22 out of 24 colonies correctly integrated donor DNA at the target sites. In other words, 92% of the colonies tested by cPCR successfully integrated the GFP sequence in place of the GAL80 open reading frame (ORF).

8.2.2. CRISPR-mediated Multiplex, Markerless, Simultaneous Genomic Integration in K. marxianus Host Cells

Several gRNAs for new loci were identified that gave high efficiency, marker-less integration in K. marxianus with CRISPR-Cas system: NDT80, YKU80, GAS2, LEU2, and GAL80. 96-well transformations were performed using the same basic protocol described above. PCR-generated linear gRNA cassettes were co-transformed with PCR-generated linear selectable marker-marked K. marxianus vector. 24 colonies of each type of 1 and 2-locus integration and 32 colonies of each type of 3-locus integrations were verified by colony PCR (cPCR). Rates of successful integration at all attempted loci were 94% at one locus, 34% at two loci and 7% at three loci. 368 colonies were screened by colony PCR. Among these colonies screened, a small proportion of cases (about 5%), no PCR fragment bands were observed on a gel, and these colonies were not included in the calculation of integration efficiencies.

TABLE 1

| | | Integration | Integration |
|---|---|---|---|
| Targeted Loci | Description of Integration | Type | Efficiency |
| GAL80 | marker-less integration with | Single | 100% |
| | simultaneous gene deletion | | |
| KU80 | marker-less integration with | Single | 88% |
| | simultaneous gene deletion | | |
| NDT80 | marker-less integration with | Single | 96% |
| | simultaneous gene deletion | | |
| GAS2 | marker-less integration with | Single | 96% |
| | simultaneous gene deletion | | |
| GAL80, | marker-less integration with | Double | 61% |
| NDT80 | simultaneous gene deletion | | |
| GAL80, GAS2 | marker-less integration with | Double | 52% |
| | simultaneous gene deletion | | |
| NDT80, GAS2 | marker-less integration with | Double | 41% |
| | simultaneous gene deletion | | |
| GAL80, KU80 | marker-less integration with | Double | 22% |
| | simultaneous gene deletion | | |
| NDT80, KU80 | marker-less integration with | Double | 38% |
| | simultaneous gene deletion | | |
| KU80, GAS2 | marker-less integration with | Double | 21% |
| | simultaneous gene deletion | | |
| GAL80, | marker-less integration with | Triple | 7% |
| NDT80, KU80 | simultaneous gene deletion | | |
| GAL80, | marker-less integration with | Triple | 0% |
| NDT80, GAS2 | simultaneous gene deletion | | |
| GAL80, | marker-less integration with | Triple | 6% |
| KU80, GAS2 | simultaneous gene deletion | | |
| NDT80, | marker-less integration with | Triple | 13% |
| KU80, GAS2 | simultaneous gene deletion | | |

### 8.2.3. High-Efficiency Multiplex Integrations in Kluyveromyces Using Meganuclease

CRISPR-Cas system can provide a highly efficient method for multiplex insertion or deletion of genes. However, each locus requires a unique gRNA, increasing the number of components required for each transformation as the number of target loci increases. F-CphI is a meganuclease that cuts a specific 24bp recognition sequence (“landing pad”). This sequence can be inserted at multiple locations in the genome. Transformation and selection for a single plasmid expressing F-CphI then leads to double-strand breaks at all recognition sites, followed by repair of these breaks by donor DNA containing homologous ends.

A K. marxianus plasmid expressing F-CphI was constructed and tested in several K. marxianus strains. This plasmid was shown to facilitate the excision of antibiotic resistance cassettes flanked by cut sites with sequence repeats and the integration of genes at one, two and three loci in pre-constructed strains. In all cases, the desired genotype was obtained with high efficiency, and colony numbers reduced as more loci were engineered simultaneously. For excision of antibiotic resistance cassettes, 100% of tested colonies successfully removed the cassette. Similarly, 100% of tested single landing-pad colonies integrated the desired DNA, 96% of double landing-pad colonies, and 20% of triple landing-pad colonies. Triple landing-pad strains produced very few colonies (<15/transformation), and were much more variable in integration efficiency (16-100% depending on the transformation).

TABLE 2

| Targeted | | Integration | Integration |
|---|---|---|---|
| Loci | Description of Integration | Type | Efficiency |
| KU80 | marker-less integration into f-CphI | Single | 99% |
| | landing pad | | |
| KU80, | marker-less integration into f-CphI | Double | 56% |
| GAS2 | landing pad | | |
| KU80, | marker-less integration into f-CphI | Triple | 8% |
| GAS2, | landing pad | | |
| GAL80 | | | |

### 8.2.4. Compatibility of SC Codon-Optimized Genes and Promoters in Kluyveromyces

Compatibility of SC codon-optimized genes and promoters were tested in K. marxianus. K. marxianus was transformed with a SC codon-optimized nucleic acid encoding fluorescent proteins operably linked by the SC pGAL1/10 bidirectional promoter. Two K. marxianus strains were made, one with the SC pGAL1/10 promoter driving GFP and RFP expression, and one with the K. marxianus pGAL1/10 promoter driving GFP and RFP expression. These constructs were integrated at the GAL80 locus (deleting the GAL80 ORF), and fluorescence was measured in a Tecan plate reader during log-phase growth. Both GFP and RFP were present at similar levels, indicating that in this case, Saccharomyces cerevisiae codon optimization and pGAL1/10 promoter expression were functional. (Data not shown).

The results shown in the Example section illustrate that Kluyveromyces is highly engineerable, allowing multiple genomic integration of heterologous nucleic acids simultaneously. Compared to CHO cells which have a doubling time of about 19-24 hours and a total genetic engineering cycling time (from one transformation to the next transformation) of about three months, Kluyveromyces has a cell population doubling time of about 2 hours and a total cycling time of about two weeks with the new stable plasmids provided in the present invention. The compositions and methods provided herein provide a large step forward in our ability to engineer Kluyveromyces for the production of new biomolecules.

### 9. Example 2

This example presents results of experiments using the stability elements in Table 3. The experiments were carried out generally as described above in Example 1, using the same strain used in Example 1 (referred to as Strain 1) and a second wild-type K. marxianus strain (referred to as Strain 2). The results show successful marker-less triple integrations with three CEN/ARS sequences (CEN/ARS_2, 3 and 5). Rates of triple integration with the CEN/ARS_2, 3 and 5 were 3%, 13% and 3% respectively (of n=30 colonies tested). Rates in Strain 1 are those directly comparable to those found in Example 1. Strain 2 had similar rates of triple integration but did not recapitulate the original data for CEN/ARS_1.

TABLE 3

| CEN/ARS | Length | Genome Location |
|---|---|---|
| CEN/ARS_1 | 1259 bp | Chr 5 Strain 1 |
| SEQ ID NO: 1 | | |
| CEN/ARS_2 | 1205 bp | Chr 6 Strain 1 |
| SEQ ID NO: 4 | | |
| CEN/ARS_3 | 2234 bp | Chr 3 Strain 1 |
| SEQ ID NO: 7 | | |
| CEN/ARS_4 | 1256 bp | Chr 5 Strain 2 |
| SEQ ID NO: 13 | | |
| CEN/ARS_5 | 232 bp | Chr 2 |
| SEQ ID NO: 14 | | |
| CEN/ARS_6 | 1157 bp | Chr 4 Strain 1 |
| SEQ ID NO: 10 | | |

### 9.1 Materials and Methods

### 9.1.1. Computational Search for Additional CEN/ARS Sequences

We searched the K. marxianus genome for three sequence elements (CDE1, CDE2, ARS) within 4000 bp of each other. Each sequence was allowed mismatches, with total homology as low at 5 bp. The percentage of A/T nucleotides was required to be greater than or equal to 60%. We identified 4 chromosomal sequences with this method (CEN/ARS_1 (SEQ ID NO: 1), CEN/ARS_2 (SEQ ID NO: 4), CEN/ARS_3 (SEQ ID NO: 7), and CEN/ARS_6 (SEQ ID NO: 10)). CEN/ARS_5 is published in Cernak and Estrela, bioRxiv doi: 10.1101/353680.

9.1.2. Strains, Gene Loci, gRNA and Donor DNA

Wild-type Strains 1 and 2 were transformed with gRNA vectors containing CEN/ARS elements to confirm CEN/ARS activity in maintaining the plasmid. For CRISPR-Cas engineering, we used the Cas9-integrated strains to perform single, double and triple integrations.

Three K. marxianus gene loci, GAS2, GAL80 and NDT80, were chosen to test the efficiency of CRISPR-Cas engineering. Their corresponding donor DNA fragments contain upstream and downstream sequences of each gene, resulting in gene knockout if successfully integrated. The gRNA sequences for each locus were as follows: GAS2 (SEQ ID NO: 16) , GAL80 (SEQ ID NO: 20) and NDT80 (SEQ ID NO: 19).

### 9.1.3. Preparation of Nucleic Acids and Transformations

Plasmid containing gRNA or donor DNA was extracted from E. coli culture by miniprep. Using miniprepped plasmid as template, high-fidelity PCR (Phusion polymerase, NEB) was carried out to amplify both gRNAs using primers SEQ ID NO: 24 and SEQ ID NO: 25 (Table 4), and donor DNA fragments were prepared as described in Section 8.1.4, above. Linear gRNA vector was generated by PCR (primers SEQ ID NO: 26 and SEQ ID NO: 27 in Table 4) followed by gel-extraction. Transformations were carried as described in Section 8.1.5, above.

TABLE 4

| SEQ ID NO | gRNA sequence | purpose |
|---|---|---|
| SEQ ID NO: 24 | CCTTATAAATCAAAAGAATAGACCGAGATAGG | gRNA amplification |
| SEQ ID NO: 25 | TGTTGTGTGGAATTGTGAGCGG | gRNA amplification |
| SEQ ID NO: 26 | GTTTTAGAGCTAGAAATAGCAAGTTAAAATAAGGC | Linear gRNA vector |
| | | PCR |
| SEQ ID NO: 27 | CGATCATTTATCTTTCACTGCGGAG | Linear gRNA vector |
| | | PCR |

### 9.2 Results

9.2.1. gRNA Vector Stability With New CEN/ARS Elements

In order to test the stability of plasmids containing different CEN/ARS elements. We transformed both Strain 1 and Strain 2 with each CEN/ARS-carrying plasmid. One plasmid that carries a PKD-1 element, as described above, was also tested. Plasmids with CEN/ARS_1, CEN/ARS_2 and CEN/ARS_5 resulted in large, round colonies with smooth edge (FIG. 3). On the other hand, plasmids with CEN/ARS_3, CEN/ARS_5 and PKD1 element gave rise to small and sectoring colonies, suggesting a weaker activity in maintaining plasmid stability. In addition, transformation with plasmid containing CEN/ARS_6 showed no colonies on the plate. This indicates that this computationally predicted CEN/ARS sequence may not have an actual activity. Similar phenotype regarding to colony shape and size was observed in both strains.

9.2.2. CRISPR-Cas Markerless Multiplex Transformation in K. marxianus

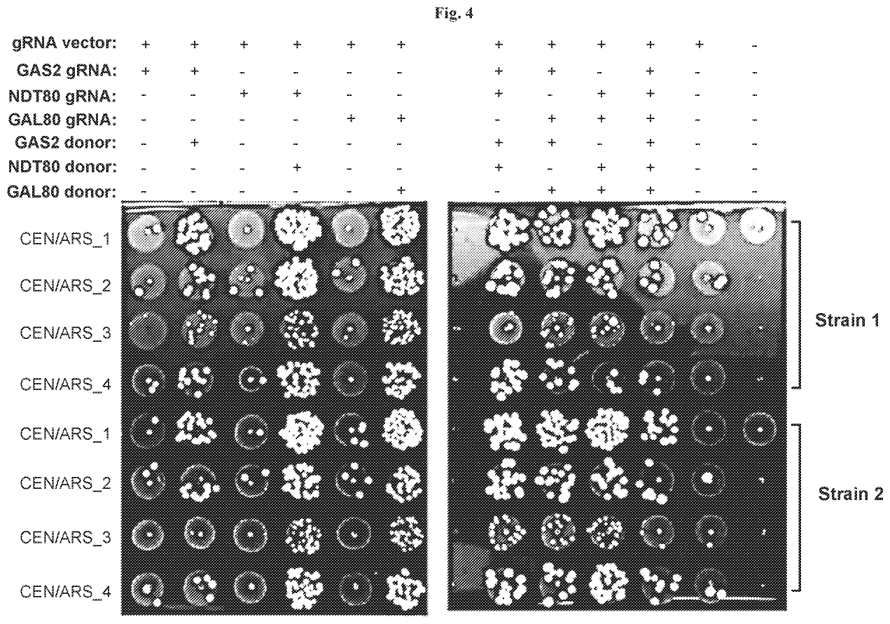

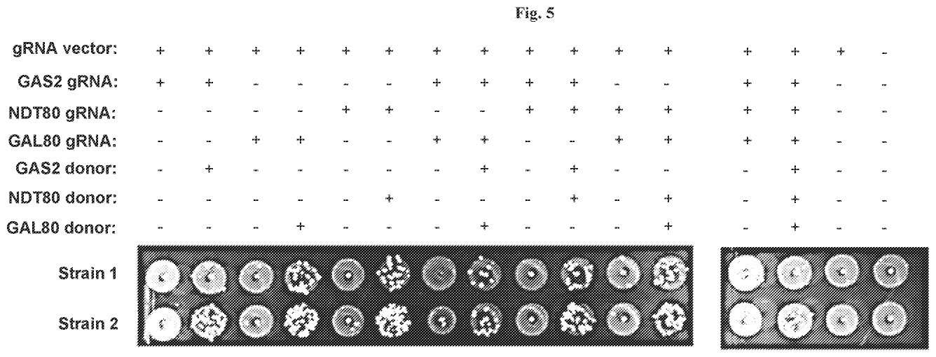

Three loci, GAS2, NDT80 and GAL80, were chosen for CRISPR-Cas-mediated integration. The 96-well transformation set-up is shown in FIGS. 4 and 5. As it is shown, strain control (no DNA construct) and plasmid control (only vector backbone) showed relatively low background colony count, as well as transformations with gRNA but without donor DNA. As the number of loci increased for simultaneous integration, fewer colonies were observed. In addition, transformation with CEN/ARS_3 resulted in small, sectoring colonies, which is in consistent with the stability experiment (FIG. 3). Similar to CEN/ARS_3, transformation with CEN/ARS_5 vector also showed small, sectoring colonies (FIG. 5).

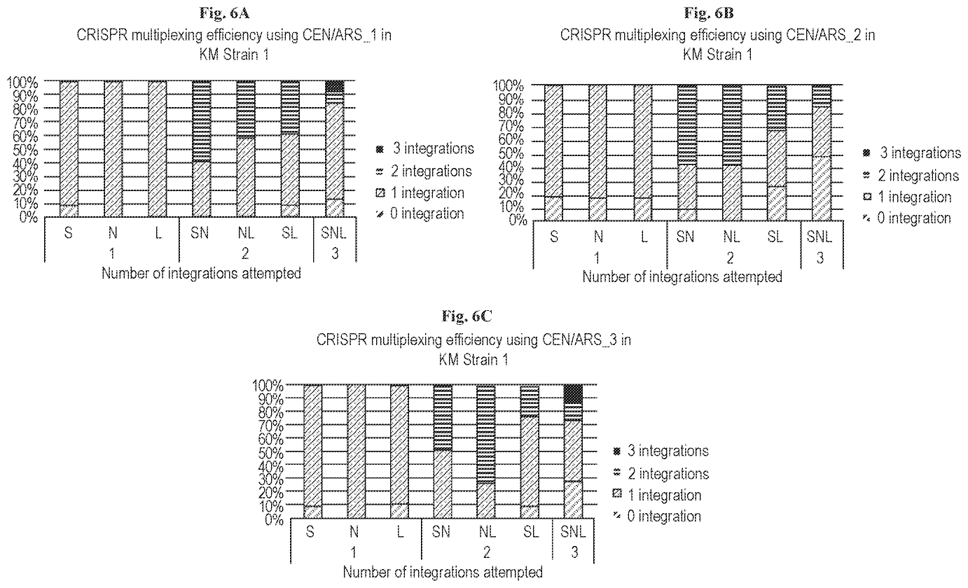

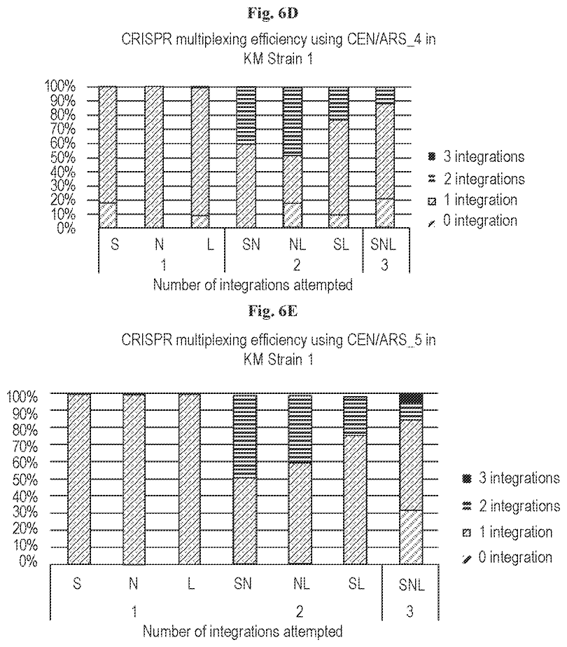

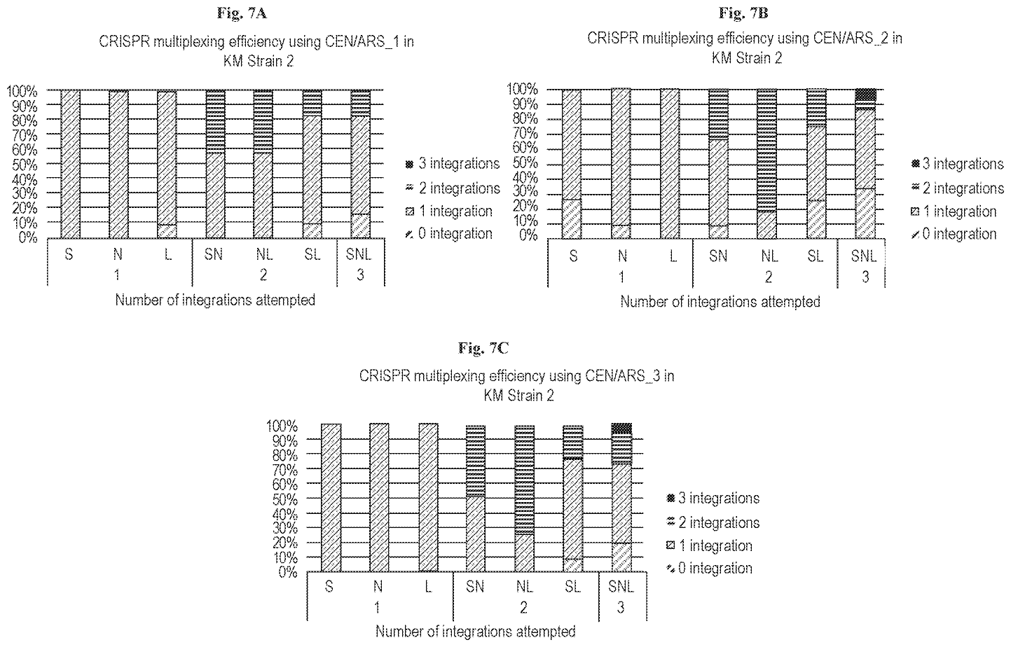

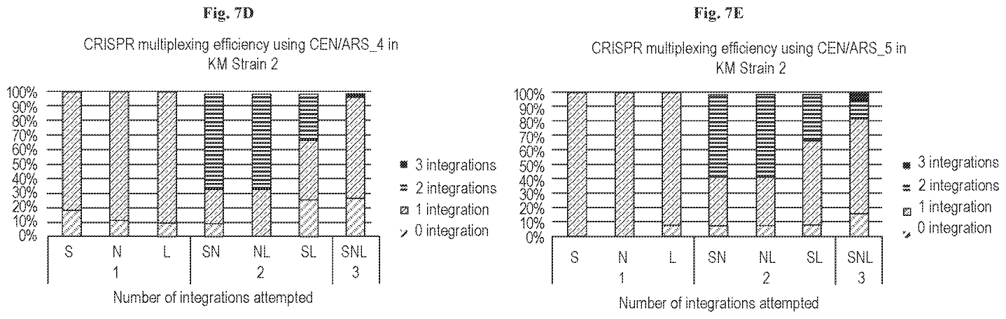

Colony PCR was performed to screen for successful integrations at each locus. For single and double integrations, 12 colonies were screened. 30 colonies were tested for triple integrations due to the low integration rate. Results for cPCR screening are shown in FIGS. 6 and 7. In general, a high rate of successful single integration ranging from 75%˜100% was observed, with vast majority of the single-locus integration rate at above 90%. Positive rate for double integration was generally from 25%˜58%. CEN/ARS_1, CEN/ARS_2, CEN/ARS_3, CEN/ARS_5 all demonstrated successful triple integrations. CEN/ARS_3 showed the highest triple integration rate at ˜13%. From the results of the stability test (FIG. 3) and CRISPR-Cas multiplexing, CEN/ARS_3 was considered to be a weak element in maintaining plasmid stability. Here the results indicates that the relationship between transformation efficiency and plasmid stability could be much more sophisticated. Besides Strain 1, Strain 2 also showed integration at all three loci at a rate ranging from 3% to 7% for CEN/ARS_2, CEN/ARS_3 and CEN/ARS_5.

In summary, we have shown that using the new CEN/ARS elements, we are able to achieve simultaneous multiplex integration in K. marxianus.

One or more features from any embodiments described herein or in the figures may be combined with one or more features of any other embodiment described herein in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Informal Sequence Listing:
SEQ ID NO: 1 (CEN/ARS 1)
Centromeric DNA elements (CDE) are underlined.
Centromere sequence (CEN) is in bold. (SEQ ID NO: 2)
ARS consensus sequence is double underlined. (SEQ ID NO: 3)

| | |
|---|---|
| | 1 11 21 31 41 |
| | GGATCCGATC TCCTTTCATT TCTGATAAAA GTAAGGCTTC TCTATTTACC |
| 51 | TTTTAACCTA CATATTCATA GTTGGAAGTT ATCCTTCTAA GTACGTATAC |
| 101 | AATATTAATT CAACGTAAAA ACAAAACTTA CTGTAAATAT GTGTAAAAAA |
| 151 | AATCTATTAA ATTCATGGCA GTTTCAAGAA AAGAAAACTA TTATGGTCTG |
| 201 | GTCACGTGTA TACAAATTAT TAATTTTAAA ACTATATAAT TTATTATTTT |
| 251 | TTTATTTTGA AGTTTAGAGT AATTTTAGTA GTATTTTATA TTTTAAATAA |
| 301 | ATATGCTTTA AATTTTTACT TAATATTTTA TTATTTTTAA ATACAACGTT |
| 351 | TTTATTTAAA ACAAAATTAT AAGTTAAAAA GTTGTTCCGA AAGTAAAATA |
| 401 | TATTTTATGG GTTTTACAAA AATAAATTAT TTTTAATGTA TTTTTTTAAT |
| 451 | TATATTTTTG TATGTAATTA TATCCACAGG TATTATGTTG AATTTAGCTG |
| 501 | TTTTAGTTTA CCTGTGTGGT ACTATGATTT TTTTAGAACT CTCCTCTTAG |
| 551 | AAATAGGTGG TGTTGCGGTT GACTTTTAAC GATATATCAT TTTCAATTTA |
| 601 | TTTATTTTAA AGTGACATAG AGAGATTCCT TTTAATTTTT TAATTTTTAT |
| 651 | TTTCAATAAT TTTAAAAATG GGGGACTTTT AAATTGGAAC AAAATGAAAA |
| 701 | ATATCTGTTA TACGTGCAAC TGAATTTTAC TGACCTTAAA GGACTATCTC |
| 751 | GAACTTGGTT CGGAAATCCT TGAAATGATT GATATTTTGG TGGATTTTCT |
| 801 | CTGATTTTCA AACAAGTAGT ATTTTATTTA ATATTTATTA TATTTTTTAC |
| 851 | 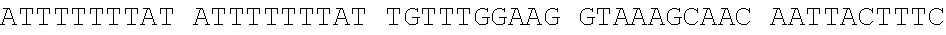 |
| 901 | AAAATATATA AATCAAACTG AAATACTTAA TAAGAGACAA ATAACATTCA |
| 951 | AGAATCAAAT ACTGGGTTAT TAATCAAAAG ATCTCTCTAC ATGCGCCCAA |
| 1001 | ATTCACTATT TAAATTTACT ATACCACTGA CAGAATATAT GAACCCAGAT |
| 1051 | TAAGTAGCCA GAGGCTCTTC CACTATATTG AGTATATAGC CTTACATATT |
| 1101 | TTCTGCGCAT AATTTACTGA TGTAAAATAA ACAAAAATAG TTAGTTTGTA |
| 1151 | GTTATGAAAA AAGGCTTTTG GAAAATGCGA AATACGTGTT ATTTAAGGTT |
| 1201 | AATCAACAAA ACGCATATCC ATAGTGGATA GTTGGATAAA ACTTCAATTG |
| 1251 | ATGCGGCCGC |

SEQ ID NO: 4 (CEN/ARS 2)
Centromeric DNA elements (CDE) are underlined.
Centromere sequence (CEN) is in bold. (SEQ ID NO: 5)
ARS consensus sequence is double underlined. (SEQ ID NO: 6)

| | |
|---|---|
| | 1 11 21 31 41 |
| | GATCCAAGTC TGAAGGTTGG TTTGGCACTA ACTTTACTCT TGTTATATTC |
| 51 | AGAATTGTAT CAAGTTTATT TGGTAGAGTG GAGCCTTTTT TTATCCGTAA |
| 101 | CACTTTTTCC CTGCTCCATT TTGAAAAACG ATTTCAGGCC ATCTTGGCTA |
| 151 | TTCCGAATGA ATTTGGAATA TGTTTAAATT AATAAAAATA AAATAAAATA |
| 201 | AAATAAAATA AAATAAAAAT TAAATCAAAT TAAATTAAAT TAAATTAAAT |
| 251 | TAAATTAAAT TAAATAAAAA TAAATACAAC CAATACAACA TGGTAATATT |
| 301 | 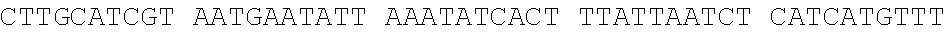 |
| 351 | 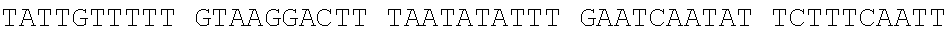 |
| 401 | ACTAGTACTT TTTTTATATG ACTAAAATTG TTACACATTG GACTGACAGT |
| 451 | AATTTTTAAA ATTTATGATT TATTCTTACT TTATATCTTT AAAAGTAGAA |
| 501 | ATATTATACG GACGCTTTGA ATACAATTGA CAACTTATCT TACTAGTGTG |
| 551 | AATCAACCCT ATCGATGTAG TACTCTTAAA ATACGGCCTT CTTGATAAAG |
| 601 | TGTTAAATTC ATTTGGGTAA TGATTTTTCG AAAACCACAT TGAATGAACG |
| 651 | ATCTAAATAA ATATAGGATG CAAAAGCATT TTAATAATTC AGAAACAAAC |
| 701 | AAATTATTAA ACAGGAGCAG TTGAACGGTA TGTTAGCGAG TTTTGTAAAG |
| 751 | GGTGAGTACA TTTATAGCTC TATTGAACAT AATAAATACA TATAAATAGT |
| 801 | ATTTTTTGAC CCTCTATGAA GATGGCTTAC CAGCAACTTA TGTCTTTTAA |
| 851 | TTCACGTGAC TACTAAACAA AAAAATATGT TATTTAAAAA ATATTTATTT |
| 901 | AAATTTTTAA ACTATTATAG ATTATTTGTG AATGCATTAT TTTTTAATTT |
| 951 | ATTAATTAAA AGAATTGCTA TTTACTTAAA ATAAGAATAA AAGCTTTTTA |
| 1001 | TTTTTTTAAA AGAAAAATAT ATTAAAAACA CTTTTCCGAA AGTTAAAATA |
| 1051 | ATTTTATATT TATCGGTAGC TGCAATTTAT AGACATAATA TTTTATATTT |
| 1101 | TTTAAAATTT ATTATTATTT TGTTTGAAAT AATAACGTCG GTGAGTGTTT |
| 1151 | AAGGTGAACT AAGACTGAAA AAGTACATAA TTTTTGTTAA TTTTATGATA |
| 1201 | TGATC |

SEQ ID NO: 7 (CEN/ARS 3)
Centromeric DNA elements (CDE) are underlined.
Centromere sequence (CEN) is in bold. (SEQ ID NO: 8)
ARS consensus sequence is double underlined. (SEQ ID NO: 9)

| | |
|---|---|
| | 1 11 21 31 41 |
| | AACAGATTGG TGGGTGGTCA ACGCACAAGC GATATCCCAA CACAGTCGGA |
| 51 | AAAACTCTCG TTCATTCCAA AACTGATTGC TTCAGATCAC AACTCCGCTG |
| 101 | GAGAAGATGA GTCCGTCACT TTCTTTCAAG ATTTGATTAA CGTTGATCGT |
| 151 | TTGAAACGTC TCAGAAATGT CACTGGTATG TCTATCGAAA TCGTGCTTGG |
| 201 | GACGCATAGA GAAATCCCAC AGCAACAGCA GCAGCAGCAG GAGTCACCTG |
| 251 | TAGCAGAAGG TGTTCCGGTC GCCCAGGATA ATGGACATGT AACCACGAAC |
| 301 | GACAATGCGG CAAATACTTC ATTGGAAGAA CCAAGTTCAC CCATTGACCA |
| 351 | GGTTTATGGA TACCTCCTAC AACAGAACAT GTCTACGTTG CCAGAAGTTA |
| 401 | CACTTTCGGA AAGTGATATC GCTATGAGCT ACCCGACGGA TCCAGTACCC |
| 451 | TCTTACAGCA GCAACTTTAA CAACTTTGCT CTGCCTACTA TTGCCGATGA |
| 501 | CAAACAAGAA TTAGAACAGA TGAGATTAAA GGAGCTAGAA AGTGAACCTC |
| 551 | CTATCTGAAC ACTTAACGAG AAATATTTAT ATGTGTGTTT TTGTTTGTAT |
| 601 | GTATGTATGT ATGTATGCCT GTGTATCATT AAATATATTA GCGGATCCCG |
| 651 | GAGTTTTTAT TATCGTGTTC TTTTCATTAT ATAGTGAACC TAAAGTGACT |
| 701 | TTCAATTCCA AATTATGGAA AGATTCCTGG CATTATGCCT TATAATAATC |
| 751 | ACTTGTTTAC AACATTCCAT TAACAACACA TGTACACTCA AATTCCATTC |
| 801 | CATAAAACCA AAAAAAACCT TATTGAATTC TCCAGACCTC TCTGTCGGCT |
| 851 | TGACTTTGCT TGCTCAATTC GCGTTTGGCT GAAGATCACT CCAGAACCTA |
| 901 | GGACGTCATT ATTGAAATCT GATCACGTGA TTCGCATATT CATATAGACG |
| 951 | TATATTTTTC GCCACTTTTC TCTCTTGAAA AAAAGTTGTG CTAGATGAAC |
| 1001 | TTTGAGAACA AAACACATTG AAAGAAAAGT GGAACATTAT AATAATTGGA |
| 1051 | AAGAATAGTA GATTGGGTGG CCAAGTGGAA GAATTTAGTA ACTTTAGTGG |
| 1101 | TTAGAGCTTG TTTGAACGAC CAATCCAGTA AACTAATCAA CCATTGAACA |
| 1151 | ATGAGTATTC CTATCTTTGG AGATCAAGTT ACCGAAGAGA GAGCAGAAAA |
| 1201 | TGCTCGTATG AGTGCCTTTG TTGGTGCCAT CGCCGTTGGT GATCTAGTGA |
| 1251 | AAACTACACT AGGTCCAAAA GGTATGGATA AGTTACTTCA AAGTGCATCC |
| 1301 | AATAGCTCGA GTTTGGTTAC AAACGATGGT GCTACCATTC TAAAATCTAT |
| 1351 | TCCTTTGGAC AACCCTGCTG CCAAGGTGCT TGTTAACATC AGTAAAGTGC |
| 1401 | AAGATGATGA AGTTGGTGAC GGTACAACAA GTGTTACTGT TCTAAGTGCA |
| 1451 | GAATTATTGA GGGAAGCTGA AAAACTTGTT GAACAAGGCA GAATTCACCC |
| 1501 | ACAAACTATC ATCGAGGGTT ACAGAATTGC TTCTGCTGCT GCCCTCTCTG |
| 1551 | CATTGGAAAA GGCTGCTGTG GACAACTCCA AGAATAAAGA AGAATTTTAC |
| 1601 | AATGATTTGA TCAGCATCGC CAACACAACG CTATCTTCTA AAATTCTATC |
| 1651 | TCAAGATAAG GCTCACTTCT CTAAGTTGGC TACCGATGCT ATCTTAAGAT |
| 1701 | TAAAGGGCTC TACGAACTTG GAACACATTC AAATTATTAA GATCATTGGT |
| 1751 | GGTAAATTAT CGGATTCTTT CCTAGATGAA GGTTTCATTT TGCCAAAGAG |
| 1801 | ATTTGGTACC AACCAACCAA AACGTGTTGA AAATGCGAAG ATTTTGATTG |
| 1851 | CCAACACTTC TCTAGATACA GACAAGGTTA AAATCTTTGG TACCAAATTT |
| 1901 | AAGGTCGACT CTACTTCCAA GTTAGCTGAA CTAGAAAAAG CTGAGCGTGA |
| 1951 | AAAAATGAAG AGAAAGATAG AAAAGATTGC ACAATTCAAC ATTAATACCT |
| 2001 | TTATCAACAG ACAATTAATC TATGACTACC CTGAACAGAT GTTTACCGAC |
| 2051 | ATGGGTATCA ACTCCATCGA ACATGCTGAC TTTGAAGGTG TTGAAAGATT |
| 2101 | AGCACTTGTC ACTGGCGGTG AGGTTGTTTC TACATTTGAC AACCCAGAAA |
| 2151 | AATGTAAGCT AGGTGAATGT AAGTTGATCG AAGAAGTTAT AATTGGTGAG |
| 2201 | GAAATCTTTA CTAAATTTAC CGGGTGCAAG TCTGGTGAAG CTTGTACCAT |
| 2251 | TGTTCTAAGG GGTGCCACTG AGCAAGTCTT GGATGAAGCA GAAAGATCTC |
| 2301 | 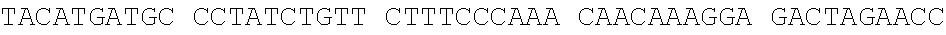 |
| 2351 | GTTCTTGGTG GTGGTTGTGC AGAAATGATA ATGTCTAAAG CAGTTGATAC |
| 2401 | TGCAGCTCAA A |

SEQ ID NO: 10 (CEN/ARS 6
Centromeric DNA elements (CDE) are underlined.
Centromere sequence (CEN) is in bold. (SEQ ID NO: 11)
ARS consensus sequence is double underlined. (SEQ ID NO: 12)

| | |
|---|---|
| | 1 11 21 31 41 |
| | TCAATTACAA AGGGTGGAAA GTGATGGGGG GAATATCATC TGCACAATTT |
| 51 | TGGCTCGCTT TATATAGTGC CGAGATTAGT AGGGTCTGGA TAAAAAAGCG |
| 101 | AAGGAGAATA GGAAGAGGAA GAAAATTTTT TTTCTTCCTC TTTGAAAGGC |
| 151 | CGGGTAACAA AGTCTCATCG TCCTCCAACC TAGGGCTTTC CTTTCCGCTT |
| 201 | TTTTTTTCTT CTTCTCCTCC AAACAAGACC CAACCATACA CACCCACACA |
| 251 | GACAGAAGAA AAAGTGTAAG GATGAGCGTT GTGTCGTTTT TTTTTTTTTT |
| 301 | TTTTTTTTTT TTGGCGGAGA ATGTGTGCAC GTGCACAGAC ACACACGGGA |
| 351 | GCGGCTGTGC CTCCGTATAC GGCAACTGCC ACGACAACCG AGGGCACAGA |
| 401 | TACACGAGGT TATGTCAAAG AGGCGTGCTG GCCTGGGGGG GGGAGGCTGC |
| 451 | GGATGCCTGA TACTGGGGCC TGATACTGAG CCCCAAGGCT CAGTCTCGGT |
| 501 | CTCTGTCTCA AGCTCAAGCC AATTCCTTCC GGGGAACCCA ACCACCTCCG |
| 551 | GATTTTTTCC GAAAGTATCC CCGAACGTCT ATGGATTATC CATGTATACA |
| 601 | 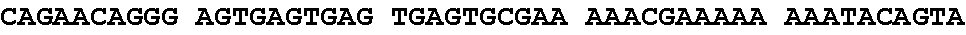 |
| 651 | 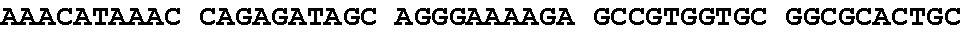 |
| 701 | GCGCCGCCCT GGGGACGGCG CCTCTCTCTA GTTCCCCCAG AAAAAAGAGT |
| 751 | CACGTGTACA CAGCCGCAGC CGCAGCCGCA GCCGCAGTAT CTCCGTGTCA |
| 801 | CATAGATTGG ACTGAACTGG ACTAGACTAG ACTAGACTAG AGAGTAGACG |
| 851 | AGAATAGACG AGACTAGACG CTCTGGCGTT TCAGATAACA CCAACACTAT |
| 901 | CTATGTTATC ATTACACACA CGATACGTAA TACGTTGGGG CTCCAGCGGT |
| 951 | CAAGGTTGGG GGTGTGGCCC ACATACGTAA CGTCTCGCCC TACACCATAC |
| 1001 | ACGGCATTTT TGTCTGCCTG CCGGCTTTGG CTTGCGCTTT GGTACTTGGT |
| 1051 | 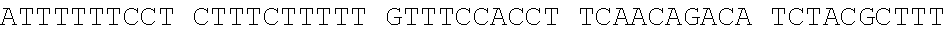 |
| 1101 | TACAGTTCAA GACATTGAAA TTTCAAGACT AGAACTAGAA TTAGAAATTG |
| 1151 | GAAATGAAAT TGGAATTATA ATAGATATTA GAAATAGATA GATATTAGAA |
| 1201 | TAGAGATAGA TATTCGAGTA ATAGAAAGGA CAAAAGTCAG GAAGAAGAAA |
| 1251 | ACTTAGGGCG AGCGAAGCTG CCGTATTAAT CTATTGGAAA ACTGAAATAC |
| 1301 | TAGGTTTCAG AGAAGAAGAA CAAACAAAAA GCGCAATAAC CAGCACTTTA |
| 1351 | TCCAAGTTAC AAGTGTGAGT GAGTGTATAT CTGCAAGCAA GGTGTGATTG |
| 1401 | AGTGAGTGAT CCGCTTGTGA TGGATTCTGT CGCTGATAGC ACCCTTGTTT |
| 1451 | CCAAAGCTGT AGCACAGCCT TCGCCGCATC ATGCTGTGAT AAAGCGTGAA |
| 1501 | CATGAGCAGG AAAGAGAAAG ACAAATAGAA GCCGAAGCAG AGGCAGAAGC |
| 1551 | AGAGGCAGAA GCAGAAGCAG AAACAGAAAT AGA |

(CEN/ARS 4)
SEQ ID NO: 13
GAGCTCCTTTCATTTCTGATAAAAGTAAGGTTTCTCTATTTATCTTTTCACCCACATTATCCTTCGAA
GTACGTATACAATATTAGTTCAACGTAAAAACAAAACTTACTGTAAATATGCGTAAAAAAAATCTATT
AAATTCATAGCAGTTTCAAGGAAAGAGAACCATTATGGTCTGGTCACGTGTGTATAAATTATTAATT
TTAACACTATATAATTTATTATTTTTTTATTTTGAAGTTTAGAGTAATTTTAGTAGTATTTTATATTTTA
AATAAATATATTTTAAATTTTTACTTAATATTTTATTATTTTTTAATACAATGTTTTTATTTAAAACAAAA
TTATAAATTAAAATGTTGTTCGAAAGTAAAATATATTTTATGGTTTTTACAAAAATAAATTATTTTTAA
TGTATTTTTTTAATTATATTTTTGTATGTAATTATATCCACAGGTATTATGTTGAATTTAGCTGTTTTA
GTTTACCTGTGTGGTACTATGAGTTTTTTGCCTCTCAAAAGCTATTTTTTAGAACTCTCTCTCCTCTT
AGAAATAGGTGGTGTTGCGGTTGACTTTTAACGATATATCATTTTCAATTTATTTATTTTAAAGTGAC
ATAGAGAGATTCCTTTTAATTTTTTAATTTTTATTTTCAATAATTTTAAAAATGGGGGACTTTTAGATT
GGAACAAAATGAAAAATATCTGTTATACGTGCAACTGAATTTTACTGACCTTAAAGGACTATCTCGA
ACTTGGTTCGGAAATCCTTGAAATGATTGATATTTTGGTGGATTTTCTCTGATTTTCAAACAAGTAGT
ATTTTATTTAATATTTATTATATTTTTTACATTTTTTTATATTTTTTTATTGTTTGGAAGGGAAAGCAAC
AATTACTTTCAAAATATATAAATTAAACTGAAATACTTAATAAGAGACAAATAACATTCAAGAATCAAA
TACTGGGTTATTAATCAAAAGATCTCTCTACATGCACCCAAATTCACTATTTAAATTTACTATACCAC
TGACAGAATATATGAACCCAGATTAAGTAGCCAGAGGCTCTTCCACTATATTGAGTATATAGCCTTA
CATATTTTCTGCGCATAATTTTCTGGATGTAAAATAAACAAAAATAGTTAGTTTGTAGTTATGAAAAA
AGGCTTTTGGAAAATGCGGAATACGTGTTATTTAAGGTTAATCAACAAAACGCATATCCATAGTGGA
TAGTTGGATAAAACTTCAATTGAT
(CEN/ARS 5)
SEQ ID NO: 14
GTCCCAGGTCTCTACAGTGAAAATATTTGCTAATTGCATACAGGAGGCTTAACTATCTCCGTTATAT
AAAAATATGAACACCCTTTTAAAACAGTTGCTGTCAACTAAATTTAGAATGTTTTTTCACTTTGGATG
AACTTTTTAATGTGATCCACTAGTTTTAATTAAATATGATTGGAAAGCACTTTTCCGTAACAAAATGA
TACAAAATGGTCAATGTTAGAAAGTACTG

| gRNA name | gRNA sequence |
|---|---|
| YYKU80_Km.gRNA_1.gg | CACGGGCAGCGCGGGGTCG SEQ ID NO: 15 |
| GAS2_Km.gRNA_0.gg | GAATCCCCCAGACCACACT SEQ |
| | ID NO: 16 |
| LEU2_Km.gRNA_0.gg | GCAGTTCCCTTGGCGTACT SEQ |
| | ID NO: 17 |
| YYKU80_Km.gRNA_0.gg | GGCCGCGGGCAACAGCCCG SEQ ID NO: 18 |
| NDT80_Km.gRNA_0.gg | GTCCGCCCAGCACAGCGCA SEQ ID NO: 19 |
| GAL80_Km.gRNA_0.gg | GCCCGGCTCAAAACCGCCC SEQ ID NO: 20 |

K. marxianus gRNA entry vector sequence
SEQ ID NO: 21
TCGCGCGTTTCGGTGATGACGGTGAAAACCTCTGACACATGCAGCTCCCGGAGACGGTCACAG
CTTGTCTGTAAGCGGATGCCGGGAGCAGACAAGCCCGTCAGGGCGCGTCAGCGGGTGTTGGCG
GGTGTCGGGGCTGGCTTAACTATGCGGCATCAGAGCAGATTGTACTGAGAGTGCAAATACACA
TCATCGTCCTACAAGTTCATCAAAGTGTTGGACAGACAACTATACCAGCATGGATCTCTTGTA
TCGGTTCTTTTCTCCCGCTCTCTCGCAATAACAATGAACACTGGGTCAATCATAGCCTACACA
GGTGAACAGAGTAGCGTTTATACAGGGTTTATACGGTGATTCCTACGGCAAAAATTTTTCATT
TCTAAAAAAAAAAAGAAAAATTTTTCTTTCCAACGCTAGAAGGAAAAGAAAAATCTAATTAAA
TTGATTTGGTGATTTTCTGAGAGTTCCCTTTTTCATATATCGAATTTTGAATATAAAAGGAGA
TCGAAAAAATTTTTCTATTCAATCTGTTTTCTGGTTTTATTTGATAGTTTTTTTGTGTATTAT
TATTATGGATTAGTACTGGTTTATATGGGTTTTTCTGTATAACTTCTTTTTATTTTAGTTTGT
TTAATCTTATTTTGAGTTACATTATAGTTCCCTAACTGCAAGAGAAGTAACATTAAAAATGAC
CACTCTTGACGACACGGCTTACCGGTACCGCACCAGTGTCCCGGGGGACGCCGAGGCCATCGA
GGCACTGGATGGGTCCTTCACCACCGACACCGTCTTCCGCGTCACCGCCACCGGGGACGGCTT
CACCCTGCGGGAGGTGCCGGTGGACCCGCCCCTGACCAAGGTGTTCCCCGACGACGAATCGGA
CGACGAATCGGACGCCGGGGAGGACGGCGACCCGGACTCCCGGACGTTCGTCGCGTACGGGGA
CGACGGCGACCTGGCGGGCTTCGTGGTCGTCTCGTACTCCGGCTGGAACCGCCGGCTGACCGT
CGAGGACATCGAGGTCGCCCCGGAGCACCGGGGGCACGGGGTCGGGCGCGCGTTGATGGGGCT
CGCGACGGAGTTCGCCCGCGAGCGGGGCGCCGGGCACCTCTGGCTGGAGGTCACCAACGTCAA
CGCACCGGCGATCCACGCGTACCGGCGGATGGGGTTCACCCTCTGCGGCCTGGACACCGCCCT
GTACGACGGCACCGCCTCGGACGGCGAGCAGGCGCTCTACATGAGCATGCCCTGCCCCTGAGT
TTAACTTGATACTACTAGATTTTTTCTCTTCATTTATAAAATTTTTGGTTATAATTGAAGCTT
TAGAAGTATGAAAAAATCCTTTTTTTTCATTCTTTGCAACCAAAATAAGAAGCTTCTTTTATT
CATTGAAATGATGAATATAAACCTAACAAAAGAAAAACAGTCGAATATCAAACATTAAAAAAA
AATAAAAGAGGTTATCTGTTTTCCCATTTAGTTGGAGTTTGCATTTTCTAATAGATAGAACTC
TCAATTAATGTGGATTTAGTTTCTCTGTTCGTTTTTTTTTGTTTTGTTCTCACTGTATTTACA
TTTCTATTTAGTATTTAGTTATTCATATAATCTTAACTTGCGGTGTGAAATACCGCACAGATG
CGTAAGGAGAAAATACCGCATCAGGAAATTGTAAGCGTTAATATTTTGTTAAAATTCGCGTTA
AATTTTTGTTAAATCAGCTCATTTTTTAACCAATAGGCCGAAATCGGCAAAATCCCTTATAAA
TCAAAAGAATAGACCGAGATAGGGTTGAGTGTTGTTCCAGTTTGGAACAAGAGTCCACTATTA
AAGAACGTGGACTCCAACGTCAAAGGGCGAAAAACCGTCTATCAGGGCGATGGCCCACTACGT
GAACCATCACCCTAATCAAGTTTTTTGGGGTCGAGGTGCCGTAAAGCACTAAATCGGAACCCT
AAAGGGAGCCCCCGATTTAGAGCTTGACGGGGAAAGCCTCTTTGAAAAGATAATGTATGATTA
TGCTTTCACTCATATTTATACAGAAACTTGATGTTTTCTTTCGAGTATATACAAGGTGATTAC
ATGTACGTTTGAAGTACAACTCTAGATTTTGTAGTGCCCTCTTGGGCTAGCGGTAAAGGTGCG
CATTTTTTCACACCCTACAATGTTCTGTTCAAAAGATTTTGGTCAAACGCTGTAGAAGTGAAA
GTTGGTGCGCATGTTTCGGCGTTCGAAACTTCTCCGCAGTGAAAGATAAATGATCGCAGTTCC
CTTGGCGTACTCGTTTTAGAGCTAGAAATAGCAAGTTAAAATAAGGCTAGTCCGTTATCAACT
TGAAAAAGTGGCACCGAGTCGGTGGTGCTTTTTTTGTTTTTTATGTCTCAGCTTTTGTTCCCT
TTAGTGAGGGTTAATTGCGCGCTTGGCGTAATCATGGTCATAGCTGTTTCCTGTGTGAAATTG
TTATCCGCTCACAATTCCACACAACATAGGAGCCGGAAGCATAAAGTGTAAAGCCTGGGGTGC
CTAATGAGTGAGGTAACTCACATTAATTGCGTTGCGCTCACTGCCCGCTTTCCAGTCGGGAAA
CCTGTCGTGCCAGGGATCCGATCTCCTTTCATTTCTGATAAAAGTAAGGCTTCTCTATTTACC
TTTTAACCTACATATTCATAGTTGGAAGTTATCCTTCTAAGTACGTATACAATATTAATTCAA
CGTAAAAACAAAACTTACTGTAAATATGTGTAAAAAAAATCTATTAAATTCATGGCAGTTTCA
AGAAAAGAAAACTATTATGGTCTGGTCACGTGTATACAAATTATTAATTTTAAAACTATATAA
TTTATTATTTTTTTATTTTGAAGTTTAGAGTAATTTTAGTAGTATTTTATATTTTAAATAAAT
ATGCTTTAAATTTTTACTTAATATTTTATTATTTTTAAATACAACGTTTTTATTTAAAACAAA
ATTATAAGTTAAAAAGTTGTTCCGAAAGTAAAATATATTTTATGGGTTTTACAAAAATAAATT
ATTTTTAATGTATTTTTTTAATTATATTTTTGTATGTAATTATATCCACAGGTATTATGTTGA
ATTTAGCTGTTTTAGTTTACCTGTGTGGTACTATGATTTTTTTAGAACTCTCCTCTTAGAAAT
AGGTGGTGTTGCGGTTGACTTTTAACGATATATCATTTTCAATTTATTTATTTTAAAGTGACA
TAGAGAGATTCCTTTTAATTTTTTAATTTTTATTTTCAATAATTTTAAAAATGGGGGACTTTT
AAATTGGAACAAAATGAAAAATATCTGTTATACGTGCAACTGAATTTTACTGACCTTAAAGGA
CTATCTCGAACTTGGTTCGGAAATCCTTGAAATGATTGATATTTTGGTGGATTTTCTCTGATT
TTCAAACAAGTAGTATTTTATTTAATATTTATTATATTTTTTACATTTTTTTATATTTTTTTA
TTGTTTGGAAGGTAAAGCAACAATTACTTTCAAAATATATAAATCAAACTGAAATACTTAATA
AGAGACAAATAACATTCAAGAATCAAATACTGGGTTATTAATCAAAAGATCTCTCTACATGCG
CCCAAATTCACTATTTAAATTTACTATACCACTGACAGAATATATGAACCCAGATTAAGTAGC
CAGAGGCTCTTCCACTATATTGAGTATATAGCCTTACATATTTTCTGCGCATAATTTACTGAT
GTAAAATAAACAAAAATAGTTAGTTTGTAGTTATGAAAAAAGGCTTTTGGAAAATGCGAAATA
CGTGTTATTTAAGGTTAATCAACAAAACGCATATCCATAGTGGATAGTTGGATAAAACTTCAA
TTGATGCGGCCGCCTGCATTAATGAATCGGCCAACGCGCGGGGAGAGGCGGTTTGCGTATTGG
GCGCTCTTCCGCTTCCTCGCTCACTGACTCGCTGCGCTCGGTCGTTCGGCTGCGGCGAGCGGT
ATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTGACGCTCAGTGGAA
CGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAG
TTACCAATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCCCCAGTGC
TGCAATGATACCGCGAGACCCACGCTCACCGGCTCCAGATTTATCAGCAATAAACCAGCCAGC
CGGAAGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGCACTGCATAA
TTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACTGATC
TTCAGCATCTTTTACTTTCACCAGCGTTTCTGGGTGAGCAAAAACAGGAAGGCAAAATGCCGC
AAAAAAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGAACGAAGCATCTGTGC
TTCATTTTGTAGAACAAAAATGCAACGCGAGAGCGCTAATTTTTCAAACAAAGAATCTGAGCT
GCATTTTTACAGAACAGAAATGCAACGCGAAAGCGCTATTTTACCAACGAAGAATCTGTGCTT
CATTTTTGTAAAACAAAAATGCAACGCGAGAGCGCTAATTTTTCAAACAAAGAATCTGAGCTG
CATTTTTACAGAACAGAAATGCAACGCGAGAGCGCTATTTTACCAACAAAGAATCTATACTTC
TTTTTTGTTCTACAAAAATGCATCCCGAGAGCGCTATTTTTCTAACAAAGCATCTTAGATTAC
TTTTTTTCTCCTTTGTGCGCTCTATAATGCAGTCTCTTGATAACTTTTTGCACTGTAGGTCCG
TTAAGGTTAGAAGAAGGCTACTTTGGTGTCTATTTTCTCTTCCATAAAAAAAGCCTGACTCCA
CTTCCCGCGTTTACTGATTACTAGCGAAGCTGCGGGTGCATTTTTTCAAGATAAAGGCATCCC
CGATTATATTCTATACCGATGTGGATTGCGCATACTTTGTGAACAGAAAGTGATAGCGTTGAT
GATTCTTCATTGGTCAGAAAATTATGAACGGTTTCTTCTATTTTGTCTCTATATACTACGTAT
AGGAAATGTTTACATTTTCGTATTGTTTTCGATTCACTCTATGAATAGTTCTTACTACAATTT
TTTTGTCTAAAGAGTAATACTAGAGATAAACATAAAAAATGTAGAGGTCGAGTTTAGATGCAA
GTTCAAGGAGCGAAAGGTGGATGGGTAGGTTATATAGGGATATAGCACAGAGATATATAGCAA
AGAGATACTTTTGAGCAATGTTTGTGGAAGCGGTATTCGCAATATTTTAGTAGCTCGTTACAG
TCCGGTGCGTTTTTGGTTTTTTGAAAGTGCGTCTTCAGAGCGCTTTTGGTTTTCAAAAGCGCT
CTGAAGTTCCTATACTTTCTAGAGAATAGGAACTTCGGAATAGGAACTTCAAAGCGTTTCCGA
AAACGAGCGCTTCCGAAAATGCAACGCGAGCTGCGCACATACAGCTCACTGTTCACGTCGCAC
CTATATCTGCGTGTTGCCTGTATATATATATACATGAGAAGAACGGCATAGTGCGTGTTTATG
CTTAAATGCGTACTTATATGCGTCTATTTATGTAGGATGAAAGGTAGTCTAGTACCTCCTGTG
ATATTATCCCATTCCATGCGGGGTATCGTATGCTTCCTTCAGCACTACCCTTTAGCTGTTCTA
TATGCTGCCACTCCTCAATTGGATTAGTCTCATCCTTCAATGCTATCATTTCCTTTGATATTG
GATCATATTAAGAAACCATTATTATCATGACATTAACCTATAAAAATAGGCGTATCACGAGGC
CCTTTCGTC
K. marxianus f-CphI plasmid sequence
SEQ ID NO: 22
TCGCGCGTTTCGGTGATGACGGTGAAAACCTCTGACACATGCAGCTCCCGGAGACGGTCACAG
CTTGTCTGTAAGCGGATGCCGGGAGCAGACAAGCCCGTCAGGGCGCGTCAGCGGGTGTTGGCG
GGTGTCGGGGCTGGCTTAACTATGCGGCATCAGAGCAGATTGTACTGAGAGTGCAAATACACA
TCATCGTCCTACAAGTTCATCAAAGTGTTGGACAGACAACTATACCAGCATGGATCTCTTGTA
TCGGTTCTTTTCTCCCGCTCTCTCGCAATAACAATGAACACTGGGTCAATCATAGCCTACACA
GGTGAACAGAGTAGCGTTTATACAGGGTTTATACGGTGATTCCTACGGCAAAAATTTTTCATT
TCTAAAAAAAAAAAGAAAAATTTTTCTTTCCAACGCTAGAAGGAAAAGAAAAATCTAATTAAA
TTGATTTGGTGATTTTCTGAGAGTTCCCTTTTTCATATATCGAATTTTGAATATAAAAGGAGA
TCGAAAAAATTTTTCTATTCAATCTGTTTTCTGGTTTTATTTGATAGTTTTTTTGTGTATTAT
TATTATGGATTAGTACTGGTTTATATGGGTTTTTCTGTATAACTTCTTTTTATTTTAGTTTGT
TTAATCTTATTTTGAGTTACATTATAGTTCCCTAACTGCAAGAGAAGTAACATTAAAAATGGG
TAAGGAAAAGACTCACGTTTCGAGGCCGCGATTAAATTCCAACATGGATGCTGATTTATATGG
GTATAAATGGGCTCGCGATAATGTCGGGCAATCAGGTGCGACAATCTATCGATTGTATGGGAA
GCCCGATGCGCCAGAGTTGTTTCTGAAACATGGCAAAGGTAGCGTTGCCAATGATGTTACAGA
TGAGATGGTCAGACTAAACTGGCTGACGGAATTTATGCCTCTTCCGACCATCAAGCATTTTAT
CCGTACTCCTGATGATGCATGGTTACTCACCACTGCGATCCCCGGCAAAACAGCATTCCAGGT
ATTAGAAGAATATCCTGATTCAGGTGAAAATATTGTTGATGCGCTGGCAGTGTTCCTGCGCCG
GTTGCATTCGATTCCTGTTTGTAATTGTCCTTTTAACAGCGATCGCGTATTTCGTCTCGCTCA
GGCGCAATCACGAATGAATAACGGTTTGGTTGATGCGAGTGATTTTGATGACGAGCGTAATGG
CTGGCCTGTTGAACAAGTCTGGAAAGAAATGCATAAGCTTTTGCCATTCTCACCGGATTCAGT
CGTCACTCATGGTGATTTCTCACTTGATAACCTTATTTTTGACGAGGGGAAATTAATAGGTTG
TATTGATGTTGGACGAGTCGGAATCGCAGACCGATACCAGGATCTTGCCATCCTATGGAACTG
CCTCGGTGAGTTTTCTCCTTCATTACAGAAACGGCTTTTTCAAAAATATGGTATTGATAATCC
TGATATGAATAAATTGCAGTTTCATTTGATGCTCGATGAGTTTTTCTAAGTTTAACTTGATAC
TACTAGATTTTTTCTCTTCATTTATAAAATTTTTGGTTATAATTGAAGCTTTAGAAGTATGAA
AAAATCCTTTTTTTTCATTCTTTGCAACCAAAATAAGAAGCTTCTTTTATTCATTGAAATGAT
GAATATAAACCTAACAAAAGAAAAAGACTCGAATATCAAACATTAAAAAAAAATAAAAGAGGT
TATCTGTTTTCCCATTTAGTTGGAGTTTGCATTTTCTAATAGATAGAACTCTCAATTAATGTG
GATTTAGTTTCTCTGTTCGTTTTTTTTTGTTTTGTTCTCACTGTATTTACATTTCTATTTAGT
ATTTAGTTATTCATATAATCTTAACTTGCGGTGTGAAATACCGCACAGATGCGTAAGGAGAAA
ATACCGCATCAGGAAATTGTAAGCGTTAATATTTTGTTAAAATTCGCGTTAAATTTTTGTTAA
ATCAGCTCATTTTTTAACCAATAGGCCGAAATCGGCAAAATCCCTTATAAATCAAAAGAATAG
ACCGAGATAGGGTTGAGTGTTGTTCCAGTTTGGAACAAGAGTCCACTATTAAAGAACGTGGAC
TCCAACGTCAAAGGGCGAAAAACCGTCTATCAGGGCGATGGCCCACTACGTGAACCATCACCC
TAATCAAGTTTTTTGGGGTCGAGGTGCCGTAAAGCACTAAATCGGAACCCTAAAGGGAGCCCC
CGATTTAGAGCTTGACGGGGAAAGCCGGCGAACGTGGCGAGAAAGGAAGGGAAGAAAGCGAAA
GGAGCGGGCGCTAGGGCGCTGGCAAGTGTAGCGGTCACGCTGCGCGTAACCACCACACCCGCC
GCGCTTAATGCGCCGCTACAGGGCGCGTCGCGCCATTCGCCATTCAGGCTGCGCAACTGTTGG
GAAGGGCGATCGGTGCGGGCCTCTTCGCTATTACGCCAGCTGGCGAAAGGGGGATGTGCTGCA
AGGCGATTAAGTTGGGTAACGCCAGGGTTTTCCCAGTCACGACGTTGTAAAACGACGGCCAGT
GAGCGCGCGTAATACGACTCACTATAGGGCGGAATAAAAAACACGCTTTTTCAGTTCGAGTTT
ATCATTATCAATACTGCCATTTCAAAGAATACGTAAATAATTAATAGTAGTGATTTTCCTAAC
TTTATTTAGTCAAAAAATTAGCCTTTTAATTCTGCTGTAACCCGTACATGCCCAAAATAGGGG
GCGGGTTACACAGAATATATAACATCGTAGGTGTCTGGGTGAACAGTTTATTCCTGGCATCCA
CTAAATATAATGGAGCCCGCTTTTTAAGCTGGCATCCAGAAAAAAAAAGAATCCCAGCACCAA
AATATTGTTTTCTTCACCAACCATCAGTTCATAGGTCCATTCTCTTAGCGCAACTACAGAGAA
CAGGGGCACAAACAGGCAAAAAACGGGCACAACCTCAATGGAGTGATGCAACCTGCCTGGAGT
AAATGATGACACAAGGCAATTGACCCACGCATGTATCTATCTCATTTTCTTACACCTTCTATT
ACCTTCTGCTCTCTCTGATTTGGAAAAAGCTGAAAAAAAAGGTTGAAACCAGTTCCCTGAAAT
TATTCCCCTACTTGACTAATAAGTATATAAAGACGGTAGGTATTGATTGTAATTCTGTAAATC
TATTTCTTAAACTTCTTAAATTCTACTTTTATAGTTAGTCTTTTTTTTAGTTTTAAAACACCA
AGAACTTAGTTTCGAATAAACACACATAAACAAACAAAATGACTAAGTTGTATTCTGACTTGT
ACAGGACCTGCATGACATGCGGAGAAGAAAAATTGTCAACCGAGTTCTACGTCAGGAACAAGA
AGACCGGAGTTAGACATTCATCATGCAAAGAGTGTGACAAGGTCAGGGTCAAATCAAGACACA
AGGAGAACCCTGAAAGGACCAAAAACAACGACTTGAAGAGATTGTACGGAATCACCTTGGACG
AGCATACCCAAATGTATGAGGAACAAAATGGTGTATGTGCAATTTGCAAGGGAGAAGGAGATG
GAAAGTGGAAGAAATTGTGTGTTGACCATGATCACGAAACAGGAAAGGTCAGGCAGTTGTTGT
GTAGGAACTGCAATATGATGTTGGGTCAGGTCAACGACAACGTTAACTTATTATCAGAAATGA
TAAAGTATTTGAAAAGATATCAGTAAAACCTGCAGGCCGCGAGCGCCGATTAAGTGAATTTAC
TTTAAATCTTGCATTTAAATAAATTTTCTTTTTATAGCTTTATGACTTAGTTTCAATTTATAT
ACTATTTTAATGACATTTTCGATTCATTGATTGAAAGCTTTGTGTTTTTTCTTGATGCGCTAT
TGCATTGTTCTTGTCTTTTTCGCCACATGTAATATCTGTAGTAGATACCTGATACATTGTGGA
TGCTGAGTGAAATTTTAGTTAATAATGGAGGCGCTCTTAATAATTTTGGGGATATTGGCTTAA
CGCGATCGCCGACGCCGCCGATGGGGGATCCACTAGTTCTAGAGCGGCCGCCACCGCGGTGGA
GCTCCAGCTTTTGTTCCCTTTAGTGAGGGTTAATTGCGCGCTTGGCGTAATCATGGTCATAGC
TGTTTCCTGTGTGAAATTGTTATCCGCTCACAATTCCACACAACATAGGAGCCGGAAGCATAA
AGTGTAAAGCCTGGGGTGCCTAATGAGTGAGGTAACTCACATTAATTGCGTTGCGCTCACTGC
CCGCTTTCCAGTCGGGAAACCTGTCGTGCCAGCTGCATTAATGAATCGGCCAACGCGCGGGGA
GAGGCGGTTTGCGTATTGGGCGCTCTTCCGCTTCCTCGCTCACTGACTCGCTGCGCTCGGTCG
TTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCT
CAAGTCAGAGGTGGCGAAACCCGACAGGACTATAAAGATACCAGGCGTTTCCCCCTGGAAGCT
CCCTCGTGCGCTCTCCTGTTCCGACCCTGCCGCTTACCGGATACCTGTCCGCCTTTCTCCCTT
CGGGAAGCGTGGCGCTTTCTCATAGCTCACGCTGTAGGTATCTCAGTTCGGTGTAGGTCGTTC
GCTCCAAGCTGGGCTGTGTGCACGAACCCCCCGTTCAGCCCGACCGCTGCGCCTTATCCGGTA
ACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAA
GGATCTTCACCTAGATCCTTTTAAATTAAAAATGAAGTTTTAAATCAATCTAAAGTATATATG
AGTAAACTTGGTCTGACAGTTACCAATGCTTAATCAGTGAGGCACCTATCTCAGCGATCTGTC
TATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTAT
CAGCAATAAACCAGCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTGGTATGGCTTCAT
TCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATTGGAAAAC
GTTCTTCGGGGCGAAAACTCTCAAGGATCTTACCGCTGTTGAGATCCAGTTCGATGTAACCCA
CTCGTGCACCCAACTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAAAGTGCCAC
CTGGGCTCGAGGATCTCCTTTCATTTCTGATAAAAGTAAGGCTTCTCTATTTACCTTTTAACC
TACATATTCATAGTTGGAAGTTATCCTTCTAAGTACGTATACAATATTAATTCAACGTAAAAA
CAAAACTTACTGTAAATATGTGTAAAAAAAATCTATTAAATTCATGGCAGTTTCAAGAAAAGA
AAACTATTATGGTCTGGTCACGTGTATACAAATTATTAATTTTAAAACTATATAATTTATTAT
TTTTTTATTTTGAAGTTTAGAGTAATTTTAGTAGTATTTTATATTTTAAATAAATATGCTTTA
AATTTTTACTTAATATTTTATTATTTTTAAATACAACGTTTTTATTTAAAACAAAATTATAAG
TTAAAAAGTTGTTCCGAAAGTAAAATATATTTTATGGGTTTTACAAAAATAAATTATTTTTAA
TGTATTTTTTTAATTATATTTTTGTATGTAATTATATCCACAGGTATTATGTTGAATTTAGCT
GTTTTAGTTTACCTGTGTGGTACTATGATTTTTTTAGAACTCTCCTCTTAGAAATAGGTGGTG
TTGCGGTTGACTTTTAACGATATATCATTTTCAATTTATTTATTTTAAAGTGACATAGAGAGA
TTCCTTTTAATTTTTTAATTTTTATTTTCAATAATTTTAAAAATGGGGGACTTTTAAATTGGA
ACAAAATGAAAAATATCTGTTATACGTGCAACTGAATTTTACTGACCTTAAAGGACTATCTCG
AACTTGGTTCGGAAATCCTTGAAATGATTGATATTTTGGTGGATTTTCTCTGATTTTCAAACA
AGTAGTATTTTATTTAATATTTATTATATTTTTTACATTTTTTTATATTTTTTTATTGTTTGG
AAGGTAAAGCAACAATTACTTTCAAAATATATAAATCAAACTGAAATACTTAATAAGAGACAA
ATAACATTCAAGAATCAAATACTGGGTTATTAATCAAAAGATCTCTCTACATGCGCCCAAATT
CACTATTTAAATTTACTATACCACTGACAGAATATATGAACCCAGATTAAGTAGCCAGAGGCT
CTTCCACTATATTGAGTATATAGCCTTACATATTTTCTGCGCATAATTTACTGATGTAAAATA
AACAAAAATAGTTAGTTTGTAGTTATGAAAAAAGGCTTTTGGAAAATGCGAAATACGTGTTAT
TTAAGGTTAATCAACAAAACGCATATCCATAGTGGATAGTTGGATAAAACTTCAATTGATGAA
TTC
yYKU70::pTEF1 > Sp.Cas9 Sequence
(SEQ ID NO: 23)
GACGGCACGGCCACGCGTTTAAACCGCCGTTATAGATGATCTTTATGACTATCACATAAATTTTGTAAC
CTTTTTCATTGGTTCAAAGGTTAAACCATTTGATGACACGACATTCGCAGATATCTTAAGGTGGGGATC
AAAAGTTAATGACACTAAAAATTGGTTATATTCTCATGGTCCAAATACAAAACCCATAAATGCATCGAC
TATTAAGTCTAAGGTCAAAAGGACAAAGGAAATAAATAGAGTGAAATTTCGCTGTCCTTTAATATTAG
ACGAAAGAGCAGACTTTGTTGTTTCTGTTAGTGGATACACAATTATATCTCATGAGATTCCTGCATCGA
AATACAAGCTTATATATGATAATGGTACGGTCAAACAAGAAGCGTACTCCCGTCGTGAATATCTTGAT
GCGGAAACTGGAGAAGTGGTACCAAATGACGAACTTGCAAAAACCTTTTCATTTGGAGATGAAATAA
TTGAGTTGTCTGAGGAAGAGAACTCACAAATTCAAAACATATACGGAAATTATGACTCATTTTTGAAG
CTAATAGGATTTAGATCTACCGAGGAATGCTTATGTTTTTACAATAATATCGACGCTCGTCCAACGCCG
GCGGACCTcgaatccttacatcacacccaatcccccacaagtgatcccccacacaccatagcttcaaaatgtttctactcctttttta
ctcttccagattttctcggactccgcgcatcgccgtaccacttcaaaacacccaagcacagcatactaaatttcccctctttcttcctcta
gggtgtcgttaattacccgtactaaaggtttggaaaagaaaaaagagaccgcctcgtttctttttcttcgtcgaaaaaggcaataaaaa
tttttatcacgtttctttttcttgaaaatttttttttttgatttttttctctttcgatgacctcccattgatatttaagttaataaacggtcttcaa
tttctcaagtttcagtttcatttttcttgttctattacaactttttttacttcttgctcattagaaagaaagcatagcaaACCTCCCGCG
ACCTCCAAAATCGAACTACCTTCACAATGGATAAGAAATACTCTATCGGTTTGGATATTGGTACTAACT
CCGTTGGTTGGGCCGTTATCACTGATGAATACAAGGTTCCATCTAAGAAGTTCAAAGTTTTGGGTAAC
ACTGATAGACACTCTATCAAGAAGAACTTGATTGGTGCTTTGTTATTTGACTCTGGTGAAACCGCTGAG
GCTACCCGTTTAAAAAGAACTGCTAGACGTAGATACACCCGTCGTAAAAACAGAATCTGTTATTTGCA
AGAGATCTTCTCCAACGAAATGGCTAAGGTTGACGACTCTTTTTTCCATAGATTAGAAGAATCTTTCTT
AGTTGAAGAAGATAAGAAGCACGAACGTCATCCAATCTTCGGTAACATTGTCGACGAAGTTGCTTACC
ATGAAAAGTACCCAACTATCTATCACTTGAGAAAGAAATTGGTTGATTCTACTGACAAAGCCGACTTG
AGATTGATCTACTTGGCTTTAGCTCATATGATCAAATTCCGTGGTCATTTTTTAATTGAAGGTGATTTGA
ACCCAGACAACTCTGACGTTGATAAATTGTTCATCCAATTGGTTCAAACCTATAACCAATTGTTTGAAG
AAAACCCAATTAACGCTTCTGGTGTTGATGCTAAGGCTATCTTGTCTGCTAGATTGTCTAAATCTAGAA
GATTGGAAAACTTAATTGCTCAATTGCCAGGTGAAAAAAAAAACGGTTTGTTCGGTAATTTGATTGCT
TTATCCTTGGGTTTGACCCCAAATTTCAAGTCCAACTTTGATTTGGCTGAAGATGCCAAGTTGCAATTG
TCTAAGGATACTTACGATGATGATTTAGATAACTTATTGGCTCAAATTGGTGATCAATACGCTGATTTG
TTTTTAGCTGCCAAGAATTTGTCCGACGCCATTTTGTTGTCTGACATCTTGAGAGTCAACACTGAAATT
ACCAAGGCCCCTTTGTCTGCTTCTATGATTAAGAGATATGACGAACACCACCAAGACTTGACCTTGTTG
AAGGCTTTGGTTAGACAACAATTACCTGAAAAGTATAAGGAAATTTTTTTCGACCAATCTAAGAACGG
TTACGCTGGTTACATTGACGGTGGTGCCTCTCAAGAAGAATTCTACAAATTCATCAAACCAATCTTGGA
AAAGATGGACGGTACTGAAGAATTGTTAGTTAAATTGAACAGAGAAGACTTGTTGAGAAAACAAAGA
ACCTTTGACAACGGTTCCATTCCTCACCAAATCCACTTGGGTGAGTTACACGCTATTTTGAGAAGACAA
GAAGATTTCTACCCATTCTTAAAGGACAACCGTGAAAAGATTGAAAAGATTTTGACCTTCAGAATTCCA
TACTACGTCGGTCCTTTGGCTCGTGGTAACTCCAGATTCGCCTGGATGACTAGAAAGTCCGAAGAAAC
TATTACTCCATGGAACTTCGAAGAAGTCGTTGACAAGGGTGCTTCTGCTCAATCCTTTATCGAAAGAAT
GACCAACTTCGACAAAAACTTGCCAAACGAAAAAGTCTTGCCAAAGCACTCTTTGTTGTATGAATACTT
TACTGTTTATAATGAATTGACTAAAGTTAAGTACGTTACTGAAGGTATGAGAAAACCAGCTTTTTTATC
TGGTGAACAAAAAAAAGCTATCGTCGATTTGTTGTTCAAAACTAACCGTAAAGTTACCGTCAAGCAAT
TGAAGGAAGATTACTTCAAGAAGATTGAATGTTTTGACTCCGTCGAAATCTCCGGTGTTGAAGACAGA
TTCAATGCTTCTTTGGGTACTTACCACGACTTGTTGAAAATTATCAAGGACAAGGATTTCTTAGATAAC
GAAGAAAACGAAGACATTTTGGAAGATATTGTCTTGACTTTGACTTTGTTCGAAGATAGAGAAATGAT
TGAAGAAAGATTGAAGACTTATGCTCATTTGTTCGACGATAAGGTCATGAAGCAATTAAAGAGAAGA
CGTTACACTGGTTGGGGTAGATTGTCTAGAAAATTGATTAACGGTATCCGTGATAAACAATCTGGTAA
GACCATCTTGGATTTCTTAAAGTCTGATGGTTTTGCCAACAGAAACTTCATGCAATTGATCCACGACGA
CTCTTTGACTTTCAAGGAGGACATTCAAAAGGCTCAAGTTTCTGGTCAAGGTGACTCTTTGCATGAACA
CATTGCCAACTTGGCTGGTTCTCCAGCTATTAAGAAGGGTATCTTGCAAACTGTTAAGGTTGTTGATGA
ATTAGTTAAGGTCATGGGTAGACACAAGCCAGAAAACATCGTCATCGAAATGGCTAGAGAAAACCAA
ACTACTCAAAAGGGTCAAAAGAATTCTAGAGAAAGAATGAAGAGAATTGAGGAAGGTATTAAGGAA
TTAGGTTCCCAAATTTTGAAGGAACATCCAGTCGAAAACACTCAATTGCAAAACGAAAAATTGTACTT
GTACTACTTACAAAACGGTAGAGATATGTATGTCGACCAAGAGTTGGACATCAACAGATTGTCCGACT
ACGATGTTGATCACATCGTTCCACAATCCTTCTTAAAGGACGACTCTATCGACAACAAGGTCTTAACCA
GATCCGACAAAAACAGAGGTAAGTCTGACAACGTTCCATCCGAAGAAGTTGTTAAAAAGATGAAGAA
CTACTGGAGACAATTGTTGAACGCCAAATTGATCACTCAAAGAAAGTTCGATAATTTGACCAAGGCTG
AAAGAGGTGGTTTGTCTGAATTGGATAAGGCTGGTTTTATTAAAAGACAATTGGTTGAGACTAGACAA
ATCACCAAGCATGTCGCTCAAATTTTAGATTCCAGAATGAACACTAAATACGACGAAAACGATAAGTT
AATTAGAGAAGTTAAGGTTATTACCTTGAAGTCTAAGTTGGTTTCTGATTTCAGAAAGGACTTCCAATT
TTACAAGGTCAGAGAAATTAACAACTACCATCACGCTCATGATGCTTACTTGAACGCCGTTGTTGGTAC
CGCTTTGATTAAAAAGTACCCAAAGTTGGAATCCGAATTTGTCTACGGTGACTACAAGGTCTACGATG
TCAGAAAAATGATCGCTAAGTCCGAACAAGAGATTGGTAAGGCTACTGCCAAGTACTTCTTTTACTCT
AACATCATGAACTTTTTCAAGACTGAAATCACTTTAGCTAACGGTGAAATTCGTAAGAGACCATTGATT
GAAACCAACGGTGAGACTGGTGAAATCGTTTGGGATAAGGGTCGTGATTTCGCTACTGTTAGAAAGG
TCTTATCTATGCCACAAGTTAACATCGTCAAGAAAACCGAAGTTCAAACTGGTGGTTTTTCTAAGGAAT
CTATCTTGCCAAAAAGAAACTCTGATAAATTGATTGCTAGAAAGAAGGATTGGGACCCAAAGAAGTAC
GGTGGTTTCGATTCCCCAACCGTCGCTTACTCCGTCTTGGTTGTCGCTAAAGTTGAAAAGGGTAAGTCC
AAGAAATTGAAGTCTGTTAAGGAATTGTTGGGTATCACTATCATGGAAAGATCTTCCTTCGAAAAGAA
CCCAATCGATTTTTTAGAGGCCAAGGGTTATAAGGAAGTTAAAAAGGACTTAATTATTAAGTTGCCAA
AGTACTCTTTGTTCGAATTAGAAAACGGTAGAAAAAGAATGTTGGCCTCTGCTGGTGAGTTGCAAAAA
GGTAACGAATTGGCCTTGCCATCTAAGTATGTTAACTTTTTGTACTTGGCCTCTCATTACGAGAAGTTG
AAGGGTTCCCCAGAAGATAACGAACAAAAGCAATTGTTCGTCGAACAACACAAACATTACTTGGATGA
AATTATCGAACAAATCTCCGAGTTTTCCAAACGTGTTATCTTGGCTGACGCCAATTTGGATAAGGTTTT
GTCTGCTTATAATAAGCATAGAGATAAGCCAATTAGAGAACAAGCCGAGAACATCATTCACTTGTTCA
CTTTGACTAATTTAGGTGCTCCAGCTGCCTTCAAATATTTCGACACCACCATTGATAGAAAGAGATACA
CCTCCACTAAGGAAGTCTTGGATGCCACCTTGATTCACCAATCTATCACTGGTTTGTACGAAACTAGAA
TCGATTTGTCTCAATTAGGTGGTGATTCCCGTGCCGACCCAAAGAAGAAGAGAAAGGTCTAAACAGG
CCCCTTTTCCTTTGTCGATATCATGTAATTAGTTATGTCACGCTTACATTCACGCCCTCCCCCCACATCCG
CTCTAACCGAAAAGGAAGGAGTTAGACAACCTGAAGTCTAGGTCCCTATTTATTTTTTTATAGTTATGT
TAGTATTAAGAACGTTATTTATATTTCAAATTTTTCTTTTTTTTCTGTACAAACGCGTGTACGCATGTAAC
ATTATACTGAAAACCTTGCTTGAGAAGGTTTTGGCATCCCCGCGTGCTTGGCCGGCCGTTTAATCAGC
GCCCAGAGACTAGCACTGAATGATCAACGGGTAGTTCACACGATGCACGAGCGCAACGCTCACAATG
ACAGTCTGGACATCAATAGTCACACTACAGAAGGTGATCTCTCAACTTCAGCAGACCATAGCGTGTAA
TAAATGCATAATTATTTTTCTCTAAAAAAAACTCAGCTGAAATTTTATATAAGTACTACATTTTATATAC
ATATTACATACTGAACAATAAGCGCGTTTGACATTTTAATTTTCGAAGACCGCGAATCCTTACATCACA
CCCAGTCCCCCAATAGTTCCCCCACACACCATGCTTCAAAAACGCACTGTACTCCTTTTTACTCTTCCGG
ATTTTCTCGGACTCTCCGCATCGCCGCACGAGCCAAGCCACACCCACACACCTCATACCATGTTTCCCCT
CTTTGACTCTTTCGTGCGGCTCCATTACCCGCATGAAACTGTATAAAAGTAACAAAAGACTATTTCGTT
TCTTTTTCTTTGTCGGAAAAGGCAAAAAAAAAAATTTTTATCACATTTCTTTTTCTTGAAAATTTTTTTTG
GGATTTTTTCTCTTTCGATGACCTCCCATTGATATTTAAGTTAATAAAAGGTCTCCCGTTTTCCAAGTTTT
AATTTGTTCCTCTTGTTTAGTCATTCTTCTTCTCAGCATTGGTCAATTAGAAAGAGAGCATAGCAAACTG
ATCTAAGTTTTAATTACCATATGAAAAAGCCTGAACTCACCGCGACGTCTGTCGAGAAGTTTCTGATCG
AAAAGTTCGACAGCGTCTCCGACCTGATGCAGCTCTCGGAGGGCGAAGAATCTCGTGCTTTCAGCTTC
GATGTAGGAGGGCGTGGATATGTCCTGCGGGTAAATAGCTGCGCCGATGGTTTCTACAAAGATCGTT
ATGTTTATCGGCACTTTGCATCGGCCGCGCTCCCGATTCCGGAAGTGCTTGACATTGGGGAATTCAGC
GAGAGCCTGACCTATTGCATCTCCCGCCGTGCACAGGGTGTCACGTTGCAAGACCTGCCTGAAACCGA
ACTGCCCGCTGTTCTGCAGCCGGTCGCGGAGGCCATGGATGCGATCGCTGCGGCCGATCTTAGCCAG
ACGAGCGGGTTCGGCCCATTCGGACCGCAAGGAATCGGTCAATACACTACATGGCGTGATTTCATATG
CGCGATTGCTGATCCCCATGTGTATCACTGGCAAACTGTGATGGACGACACCGTCAGTGCGTCCGTCG
CGCAGGCTCTCGATGAGCTGATGCTTTGGGCCGAGGACTGCCCCGAAGTCCGGCACCTCGTGCACGC
GGATTTCGGCTCCAACAATGTCCTGACGGACAATGGCCGCATAACAGCGGTCATTGACTGGAGCGAG
GCGATGTTCGGGGATTCCCAATACGAGGTCGCCAACATCTTCTTCTGGAGGCCGTGGTTGGCTTGTAT
GGAGCAGCAGACGCGCTACTTCGAGCGGAGGCATCCGGAGCTTGCAGGATCGCCGCGGCTCCGGGC
GTATATGCTCCGCATTGGTCTTGACCAACTCTATCAGAGCTTGGTTGACGGCAATTTCGATGATGCAGC
TTGGGCGCAGGGTCGATGCGACGCAATCGTCCGATCCGGAGCCGGGACTGTCGGGCGTACACAAATC
GCCCGCAGAAGCGCGGCCGTCTGGACCGATGGCTGTGTAGAAGTACTCGCCGATAGTGGAAACCGAC
GCCCCAGCACTCGTCCGAGGGCAAAGGAATAGGGAAATTGATAAGACTTTTCTAGTTGCATATCTTTT
ATATTTAAATCTTATCTATTAGTTAATTTTTTGTAATTTATCCTTATATAGTCTGGTTATTCTAAAATATC
ATTTCAGTATCTAAAATAGTTCTTTTTTTTTTTGAGTTAGATTTTTATGGGGGAGAGTTGAAGTGTTGAA
TTTTCCCACTTTGCTTCGGGATTGTGGGTCATTCTGTCGATAACTGATATCACATCATCAATAGAACCTC
TTAGATGCACGAGCGCAACGCTCACAATTAATCAGCGCCCAGAGACTAGCACTGAATGATCAACGGG
TAGTTCACACAGGTCCGCCGGCGTTGGACGAGCGCTATCGTATACCATTTATAGATGAAGTCAGGAAA
CTACCTACTTTATCGAGCTATCCAGAACTACTAGAAAGTGATGATTATCAAGTACTCAGTAGAGTCACT
GAAACGCTCGTGAATTTTTTCAATTTGAAAAATGGGTACAAGCCTTCTGATTACCACAGCCCAGCGCTT
CAAAGACACTTCACGGTACTCAGAGAGTATCTTCTCCAGATTGAAAGTAAGGAAACTAAAGATCAAGA
TGAAGATGACGAAACTCTTCTGAAAGTCAAACAGATTCACGAAAGAATTGCTGCTTCTGCTCAATCAG
ATGATCCTAAACAGCAAAGACTAGTAAAGTATTTGAAACTATGGAATTCATATTACAATCGCTATAATA
ATTTGGAAATTGAATCAAAACCAAAACAGAATAAACGGAGTAAATTTAATATATAATATATAATAATA
TTCTATCGGCGGTTTAAACGCGTGGCCGTGCCGTC

What is claimed:

1. A method for modifying a target site in a Kluyveromyces host cell genome, the method comprising:
(a) contacting the host cell, which has reduced non-homologous end joining (NHEJ) activity, with:
(i) a nucleic acid molecule comprising a stability element comprising a centromere sequence (CEN) sequence at least 95% identical to SEQ ID NO: 2 and an autonomously replicating sequence (ARS) consensus sequence at least 90% identical to SEQ ID NO: 3 and nucleic acid sequence encoding a nuclease capable of cleaving the target site; and
(ii) a donor DNA molecule capable of homologous recombination at the cleaved target site;
(b) selecting a transformed host cell in which the donor DNA molecule integrated into the target site.

2. The method of claim 1, wherein the host cell is K. marxianus.

3. The method of claim 1, wherein the nuclease is a meganuclease.

4. The method of claim 3, wherein the meganuclease is F-CphI.

5. The method of claim 1, wherein the stability element is at least 90% identical to a sequence less than 750 bp in length and comprising residues 202 to 876 of SEQ ID NO: 1.

6. The method of claim 1, wherein the stability element is at least 95% identical to SEQ ID NO: 1.

7. The method of claim 1, wherein the step of contacting includes contacting the host cell with two or more donor DNA molecules capable of homologous recombination with different target sites in the host cell genome, whereby homologous recombination in the host cell results in integration of the donor DNA molecules at the different target sites.

8. A host cell made by the method of claim 1.

9. A recombinant nucleic acid molecule comprising:
(i) a nucleic acid sequence encoding a nuclease or a sequence encoding a crRNA activity and a tracrRNA activity that enables site-specific recognition and cleavage of a target site by an RNA-guided DNA endonuclease; and
(ii) a stability element comprising a centromere sequence (CEN) sequence at least 95% identical to SEQ ID NO: 2 and an autonomously replicating sequence (ARS) consensus sequence at least 90% identical to SEQ ID NO: 3.

10. The recombinant nucleic acid molecule of claim 9, wherein the stability element is at least 95% identical to a sequence less than 750 bp in length and comprising residues 202 to 876 of SEQ ID NO: 1.

11. The recombinant nucleic acid molecule of claim 9, wherein the stability element is at least 95% identical to SEQ ID NO: 1.

12. The recombinant nucleic acid molecule of claim 9, wherein the nuclease is an RNA-guided DNA endonuclease.

13. The recombinant nucleic acid molecule of claim 12, wherein the RNA-guided DNA endonuclease is a Cas9 endonuclease.

14. The recombinant nucleic acid molecule of claim 9, wherein the nuclease is a meganuclease.

15. The recombinant nucleic acid molecule of claim 14, wherein the meganuclease is F-CphI.
